(19) United States
(12) Patent Application Publication
(10) Pub. No.: US 2007/0213263 A1
(43) Pub. Date: Sep. 13, 2007

(54) Methods And Compositions Using Adiponectin For Treatment Of Cardiac Disorders And For Stimulation Of Angiogenesis

(75) Inventors: Kenneth Walsh, Carlisle, MA (US); Noriyuki Ouchi, Brookline, MA (US)

Correspondence Address: Ronald I Eisenstein;Nixon Peabody, 100 Summer Street, Boston, MA, 02110, US

(73) Assignee: The Trustees of Boston University, Boston, MA (US)

(21) Appl. No.: 10/574,828

(22) PCT Filed: Oct. 8, 2004
(86) PCT No.: PCT/US04/33178

§ 371 Date: Dec. 27, 2006

Related U.S. Application Data
(60) Provisional application No. 60/510,057, filed on Oct. 9, 2003.

(51) Int. Cl. A61K 38/17; A61K 48/00

(52) U.S. Cl. 514/12; 514/44

(57) ABSTRACT

We have surprisingly discovered that adiponectin regulates angiogenesis and have shown that adiponectin is an effective agent at treating cardiac disorders. As a result of this discovery, the present invention provides methods for treatment of cardiac disorders and methods for stimulation of angiogenesis in tissues using adiponectin. In one preferred embodiment, the methods of the invention are used to treat complications of diabetes, such as ischemic limbs and hypertrophic cardiomyopathy.

### CROSS REFERENCE

This Application claims the benefit under 35 U.S.C §119(e) of U.S. Provisional Application No. 60/510,057, filed Oct. 9, 2003.

### FIELD OF THE INVENTION

The present invention provides for novel methods for treatment of cardiac disorders and for treatment of diseases or disorders where stimulation of angiogenesis is desired, and related compounds.

### BACKGROUND OF THE INVENTION

Adipose tissue secretes various bioactive substances, referred to as adipocytokines, whose dysregulation directly contributes to obesity-related diseases¹⁻⁴. Adiponectin/ACRP30 is an adipocytokine that is abundantly present in plasma^{5,6}, but is downregulated in association with obesity-linked diseases including coronary artery diseases,^{7,8} type 2 diabetes⁹ and hypertension.^{53,58} Adiponectin inhibits monocyte adhesion to endothelial cells⁷, macrophage transformation to foam cells¹⁰, and vascular smooth muscle cell proliferation¹¹ in vitro. Adiponectin-knockout (APN-KO) mice exhibit diet-induced insulin resistance, increased intimal hyperplasia in response to acute vascular injury and impaired endothelium-dependent vasodilatation in response to an atherogenic diet^{53,59,60}, Conversely, forced adiponectin expression reduces atherosclerotic lesions in a mouse model of atherosclerosis and has anti-inflammatory effects on the vasculature,¹² whereas adiponectin-deficient mice exhibit excessive vascular remodeling response to acute injury¹³ and diet-induced insulin resistance¹⁴. Therefore, adiponectin is considered a biologically relevant modulator of vascular remodeling with anti-atherogenic and anti-diabetic properties.

Obesity is strongly associated with the metabolic syndrome, type 2 diabetes, hypertension and heart disease^{52.53}. Adipose tissue may function as an endocrine organ by secreting adipocytokines that can directly or indirectly affect obesity-linked disorders^{53,54}. Pathologic cardiac remodeling characterized by myocardial hypertrophy occurs with many obesity-related conditions^{55,56}, and diastolic dysfunction is one of the earliest clinical manifestations of insulin resistance or diabetes⁵⁷. However, the molecular links between obesity and cardiac remodeling have not been clarified.

Vascular endothelial cells are in direct contact with plasma and play pivotal roles in angiogenesis and maintaining whole body homeostasis^{15,16}. Dysregulated angiogenesis is a characteristic of obesity-related disorders including atherosclerosis, diabetes, and hypertension¹⁷. However, an interaction between adiponectin and angiogenesis has not been elucidated.

Inappropriate angiogenesis can have severe negative consequences. For example, it is only after many solid tumors are vascularized as a result of angiogenesis that the tumors have a sufficient supply of oxygen and nutrients that permit it to grow rapidly and metastasize. Therefore, maintaining the rate of angiogenesis in its proper equilibrium is critical to a range of functions, and it must be carefully regulated.

The rate of angiogenesis involves a change in the local equilibrium between positive and negative regulators of the growth of microvessels. The therapeutic implications of angiogenic growth factors were first described by Folkman and colleagues over two decades ago⁴⁷. Abnormal angiogenesis occurs when there are either increased or decreased stimuli for angiogenesis resulting in excessive or insufficient blood vessel growth, respectively. For instance, conditions such as ulcers, strokes, and heart attacks may result from the absence or lower levels of angiogenesis than normally required for natural healing.

Thus, there are instances where a greater degree of angiogenesis is desirable. For example, investigations have established the feasibility of using recombinant angiogenic growth factors, such as fibroblast growth factor (FGF) family^{48,49}, endothelial cell growth factor (ECGF)⁵⁰, and more recently, vascular endothelial growth factor (VEGF) to expedite and/or augment collateral artery development in animal models of myocardial and hindlimb ischemia^{50,51}. Stimulation of angiogenesis would also increase blood circulation and aid in wound and ulcer healing. In one highly desirable aspect, angiogenesis stimulators can be used for treatment of heart conditions, such as myocardial infarction and cardiac myopathy.

Although preliminary results with the angiogenic proteins are promising, new angiogenic agents that show improvement in size, ease of production, stability and/or potency would be desirable. In particular, it is highly desirable to find agents that can effectively treat cardiac disorders. Heart failure is one of the leading causes of morbidity and mortality in the world. In the U.S. alone, estimates indicate that 3 million people are currently living with cardiomyopathy and another 400,000 are diagnosed on a yearly basis.

### SUMMARY OF THE INVENTION

We have surprisingly discovered that adiponectin, an adipocyte specific cytokine, regulates angiogenesis. We have further shown that adiponectin is an effective agent in treating cardiac disorders, e.g. cardiac hypertrophy. As a result of our discoveries, the present invention provides for use of adiponectin to stimulate angiogenesis in situations where angiogenesis is desired and further provides methods for treatment of cardiac disorders with adiponectin (e.g. myocardial infarction or cardiac hypertrophy).

The present invention provides methods for stimulating angiogenesis in a tissue associated with a condition or disorder where angiogenesis is needed. A composition comprising an angiogenesis-stimulating amount of adiponectin protein or a nucleic acid encoding such protein is administered to tissue to be treated for a disease condition or disorder that responds to new blood vessel formation.

The composition providing the adiponectin protein can contain purified protein, biologically active protein fragments such as an angiogenesis promoting fragment (or as discussed below a cardiac treating fragment), recombinantly produced adiponectin protein or protein fragments or fusion proteins, or gene/nucleic acid expression cassettes for expressing adiponectin protein. Such a cassette contains the gene operably linked to a promoter capable of expressing the gene n the desired tissue. As explained below, the promoter is preferably inducible, e.g. TetR linked to a TetR by an IRES. The cassette can be delivered by known means including vectors, catheters, gene gun, etc.

The tissue to be treated can be any tissue in which potentiation of angiogenesis is desirable. For example, adiponectin is useful to treat patients with hypoxic tissues such as those following stroke, myocardial infarction or associated with chronic ulcers, tissues in patients with ischemic limbs in which there is abnormal, i.e., poor circulation, due to diabetic or other conditions. Patients with chronic wounds that do not heal, and therefore could benefit from the increase in vascular cell proliferation and neovascularization, can be treated as well. Potentiation of angiogenesis would also offer therapeutic benefit for ischemic vascular diseases, including coronary artery insufficiency and ischemic cardiomyopathy, peripheral arterial occlusive disease, cerebrovascular disease, ischemic bowel syndromes, impotence, and would healing.

The adiponectin protein, peptide, and nucleic acid sequence encoding adiponectin protein or peptide may be administered in conjunction with another angiogenesis stimulator.

The present invention also provides a method for treating a cardiac disorder comprising administering to a patient having said disorder a pharmaceutical composition comprising adiponectin protein or a nucleotide sequence encoding for said protein.

In one embodiment, the cardiac disorder is associated with abnormal circulation, for example, a myocardial infarction or ischemic vascular diseases including, but not limited to, coronary artery insufficiency and ischemic cardiomyopathy, peripheral arterial occlusive disease, and cerebrovascular disease.

In one embodiment, the patient having said cardiac disorder is diabetic.

In one embodiment, the patient having said cardiac disorder is not diabetic.

In one embodiment, the cardiac disorder is cardiac hypertrophy.

In another embodiment, the cardiac disorder is cardiomyopathy.

The cardiac disorder to be treated by methods of the invention, may or may not be associated with abnormal circulation. For example, cardiac hypertrophy.

The adiponectin protein, peptide, and nucleic acid sequence encoding adiponectin protein or peptide may be administered in conjunction with other agents known to treat cardiac disorders.

The present invention further encompasses kits for treating such conditions. The kits can contain pharmaceutical compositions comprising a viral or non-viral gene transfer vector containing a nucleic acid, the nucleic acid having a nucleic acid segment encoding for adiponectin protein or peptide, and a pharmaceutically acceptable carrier that are suitable for stimulating angiogenesis in a target mammalian tissue and/or treating a cardiovascular disorder. The kit can also contain the adiponectin protein or biologically effective portion thereof.

Other aspects of the invention are disclosed infra.

### BRIEF DESCRIPTION OF THE DRAWINGS

FIGS. 1A to 1C show that adiponectin promotes endothelial cell migration and differentiation into tube-like structures. Tube formation assays were performed (FIG. 1A and FIG. 1B). HUVECs were seeded on Matrigel-coated culture dishes in the presence of adiponectin (30 μg/ml), VEGF (20 ng/ml) or BSA (30 μg/ml)(Control). FIG. 1A) Representative cultures are shown. FIG. 1B) Quantitative analysis of tube formation. FIG. 1C) A modified Boyden chamber assay was performed using HUVECs. HUVECs were treated with adiponectin (30 μg/ml), VEGF (20 ng/ml) or BSA (30 μg/ml)(Control). Results are show as the mean±SE. Results are expressed relative to the values compared to control. *p<0.01 vs. control.

FIGS. 2A to 2C show adiponectin-stimulated signaling in endothelial cells. FIG. 2A) Time-dependent changes in the phosphorylation of AMPK, Akt, eNOS and ERK following adiponectin treatment (30 μg/ml). FIG. 2B) Role of AMPK in the regulation of adiponectin-induced protein phosphorylation. HUVECs were transduced with an adenoviral vector expressing dominant-negative AMPK tagged with c-Myc (dn-AMPK) or an adenoviral vector expressing GFP (Control) 24 h before serum-starvation. After 16-h serum-starvation, cells were treated with adiponectin (30 μg/ml) for the indicated lengths of time. FIG. 2C) Role of Akt in the regulation of adiponectin-induced protein phosphorylation. HUVECs were transduced with an adenoviral vector expressing dominant-negative Akt (dn-Akt) or an adenoviral vector expressing GFP (Control) 24 h before serum-starvation. After 16-h serum-starvation, cells were treated with adiponectin (30 μg/ml) for the indicated lengths of time. Representative blots are shown.

FIGS. 3A to 3C show the contribution of AMPK and Akt to adiponectin-induced angiogenic cellular responses. HUVECs were transduced with an adenoviral vector expressing dn-AMPK (hatch), dn-Akt (open) or GFP (Control, solid) 24 h before the change to low-serum media. After 16-h serum-starvation, in vitro Matrigel (FIG. 3A, FIG. 3B) or modified Boyden chamber assays (FIG. 3C) were performed. Cells were treated with adiponectin (30 μg/ml) or BSA (30 μg/ml)(Vehicle). A) Representative cultures displaying tube formation are shown. FIG. 3B) Quantitative analysis of tube lengths. FIG. 3C) Modified Boyden chamber assay was performed with adiponectin or VEGF as chemoattractant. Results are shown as the mean±SE. Results are expressed relative to the values compared to control. *p<0.01 vs. each control.

FIGS. 4A to 4C shows that PI3-kinase signaling is involved in adiponectin-induced angiogenic pathway. FIG. 4A) Quantitative analysis of tube formation is shown. HUVECs were treated with adiponectin (30 μg/ml) or BSA (30 μg/ml) in the presence of LY294002 (10 μM) or vehicle at the time seeding. FIG. 4B) A modified Boyden chamber assay was performed using adiponectin as the chemoattractant. HUVECs were pretreated with LY294002 (10 μm) or vehicle for 1 h and then incubated with adiponectin (30 μg/ml) or BSA (30 μg/ml) for 4 h. FIG. 4C) Effects of LY294002 on adiponectin-stimulated protein phosphorylation. Representative blots are shown. HUVECs were pretreated with LY294002 (10 μM) or vehicle for 1 h and then incubated with adiponectin (30 μg/ml) or BSA (30 μg/ml) for the indicated lengths of time. Results are presented as the mean±SE. For A and B, results are expressed relative to the values compared to control. *, p<0.01.

FIGS. 5A to 5D show that adiponectin promotes angiogenesis in vivo. An in vivo Matrigel plug assay was performed to evaluate the effect of adiponectin on angiogenesis (FIG. 5A and FIG. 5B). Matrigel plugs containing adiponectin (100 μg/ml, n=3) or PBS (Control, n=3) were injected subcutaneously into mice. A) Plugs were stained with the endothelial cell marker CD31. Bar: 100 μm. FIG. 5B) The frequency of CD31-positive cells in five low power fields was determined for each Matrigel plug. Data were presented as fold increase of CD31-positive cells relative to the control. Rabbit cornea assay was performed (FIG. 5C and FIG. 5D). Pellets containing adiponectin (1 μg and 10 μg, n=8), VEGF (100 ng, n=8) or PBS (Control, n=8) were implanted in the cornea. FIG. 5C) Photographs of rabbit eyes are shown (Control, adiponectin 10 μg, VEGF 100 ng). FIG. 5D) An angiogenic score was calculated (vessel density×distance from limbus). Results are shown as the mean±SE. *P<0.01 vs. control.

FIG. 6 shows a proposed scheme for adiponectin-stimulated signaling in endothelial cells. Adiponectin activates AMPK which, in turn, promotes Akt activation, eNOS phosphorylation and angiogenesis. PI3-kinase is essential for adiponectin-mediated activation of Akt. Both AMPK and Akt can directly phosphorylate eNOS. However, inhibition of Akt or PI3-kinase was found to suppress adiponectin-stimulated eNOS phosphorylation without interfering with AMPK activation. Therefore, the data are most consistent with an AMPK-PI3-kinase-Akt-eNOS signaling axis.

FIG. 7 shows a table of body weight and echocardiographic measurements in WT and APN-KO mice at 7 days post-surgery.

FIGS. 8A to 8J shows enhanced pressure overload-induced cardiac hypertrophy in adiponectin-KO mice subjected to transverse aortic constriction (TAC). WT mice. (FIG. 8A, left) Representative pictures of hearts from WT and APN-KO mice at 7 days after sham operation or TAC. (FIG. 8A, right) Representative hematoxylin and eosin-stained cross-sections of left ventricular myocardium from WT and APN-KO mice at 7 days after sham operation or TAC. (FIG. 8B) Representative M-mode echocardiogram for APN-KO and WT mice at 7 days after sham operation or TAC. (FIG. 8C) HW/BW ratio in WT (n=6) and KO mice (n=5) at 7 days after sham operation or TAC. (FIG. 8D) Histological analysis of heart sections from WT and APN-KO mice stained with Masson's trichrome. (×400; bar indicates 50 μm). Quantitative analysis of cardiac myocyte cross-sectional area (n=200 per section) in WT (n=6) and APN-KO mice (n=5). (FIG. 8E) Decreased survival of adiponectin-KO (APN-KO) mice (closed square) after TAC (n=20) (*P<0.05, **P<0.01) in comparison with wild-type (WT) mice (closed circle) after TAC (n=20). Adenovirus-mediated supplementation of adiponectin in APN-KO (n=9) (open circle) improves survival to a level that is comparable to that of wild type. (FIG. 8F) Oligomeric state of adenovirus-delivered adiponectin in APN-KO mouse (open circle) and endogenous adiponectin in WT mouse (closed circle) assessed by gel filtration analysis. The adenoviral vector expressing adiponectin (Ad-APN, 2×10⁸ pfu total) was delivered intravenously via the jugular vein, and the oligomeric state of adiponectin was analyzed 3 days after Ad-APN injection. (FIG. 8G) Adenovirus-mediated supplementation of adiponectin in APN-KO and WT mice attenuates cardiac hypertrophy in response to TAC mice as shown by echocardiography. Adenoviral vectors expressing adiponectin (Ad-APN, 2×10⁸ pfu total, n=3) or β-galactosidase (control, n=3) were delivered intravenously via the jugular vein 3 days before TAC surgery. LV wall thickness (IVS and LVPW) was determined at 3 days after TAC. (FIG. 8H) HW/BW ratio and cardiac myocyte cross-sectional area in WT (n=5) and KO mice (n=3) treated with Ad-APN or Ad-βgal (control) were determined at 7 days after sham operation or TAC. (FIG. 8I) Adenovirus-mediated supplementation of adiponectin in diabetic db/db mice attenuates cardiac hypertrophy in response to TAC as shown by echocardiography. Ad-APN (2×10⁸ pfu total, n=4) or β-galactosidase (control, n=4) were delivered intravenously via the jugular vein 3 days before TAC surgery. Wall thickness (IVS and LVPW) was determined at 3 days after TAC surgery or sham operation. (FIG. 8J) APN-KO mice display an increased cardiac hypertrophy following AngII infusion relative to WT mice (n=4). Adenovirus-mediated supplementation of adiponectin (2×10⁸ pfu) in APN-KO (n=4) and WT (n=4) mice attenuates AngII-induced cardiac hypertrophy. Wall thickness (IVS and LVPW) was determined after 14 days of AngII infusion.

FIGS. 9A to 9E show that adiponectin inhibits the hypertrophic response to α-adrenergic receptor (αAR) stimulation or pressure overload. (FIG. 9A) Representative example of immunostaining of sarcomeric F-actin with rhodamine phalloidin in rat cardiac myocytes. Cells were pretreated with adiponectin (30 μg/ml) or vehicle for 30 min, propranolol (Pro; 2 μM) for an additional 30 min, followed by the addition of norepinephrine (NE) for 48 hours. (FIG. 9B) Quantitative analysis of cell surface area measured by semi-automatic computer-assisted planimetry (Bioquant) from two-dimensional images of 100 cells selected at random (left panel) and protein synthesis measured by [³H] leucine incorporation (right panel). (FIG. 9C) The phosphorylation (P−) of ERK in heart tissues from WT and APN-KO mice at 7 days after sham operation or TAC. (FIG. 9D) Effect of adiponectin on the phosphorylation of ERK in response to αAR-stimulation in cultured rat cardiac myocytes. Cells were pretreated with adiponectin (30 μg/ml) or vehicle for 30 minutes, 2 μM Pro for an additional 30 minutes and then stimulated with or without 1 μM NE for the indicated lengths of time. (FIG. 9E) Effects of three different oligomeric forms of adiponectin on the phosphorylation of ERK in response to αAR-stimulation in cultured rat cardiac myocytes. Cells were pretreated with each form of adiponectin (5 μg/ml) or vehicle for 30 minutes, 2 μM Pro for an additional 30 minutes and then stimulated with 1 μM NE for 5 minutes. Relative phosphorylation levels of ERK were quantified using NIH image program. Immunoblots were normalized to total loaded protein. *p<0.05 vs. WT. **p<0.05 vs. control.

FIGS. 10A to 10F show adiponectin inhibition αAR-stimulated myocyte hypertrophy is mediated via AMPK signaling. (FIG. 10A) Time-dependent changes in the phosphorylation of AMPK in rat cultured cardiac myocytes after adiponectin treatment (30 μg/ml). (FIG. 10B) Effects of three different oligomeric forms of adiponectin (5 μg/ml) on the phosphorylation of AMPK. (FIG. 10C) The phosphorylation of AMPK in myocardium from WT and APN-KO mice at 7 days after sham operation or TAC. (FIG. 11D) Ad-dnAMPK reverses adiponectin stimulation of AMPK and ACC phosphorylation. Rat cardiac myocytes were transduced with c-myc-tagged Ad-dnAMPK or Ad-βgal (control) at a multiplicity of infection of 50 for 24 hours in serum starved media. Cells were treated with adiponectin (30 μg/ml) for the indicated lengths of time. (FIG. 10E) Contribution of AMPK signaling to the inhibitory effect of adiponectin on αAR-stimulated myocyte hypertrophy. After 24-hour transduction of rat cardiac myocytes with Ad-dnAMPK or Ad-βgal (control), cells were pretreated with adiponectin (30 μg/ml) or vehicle for 30 minutes then treated with 2 μM Pro for 30 minutes and stimulated with or without 1 μM NE for 48 hours. Quantitative analysis of cell surface area was performed in 100 randomly selected cells (left panel) or ³H-leucine incorporation into protein (right panel). (FIG. 10F) Effect of Ad-dnAMPK on adiponectin inhibition of NE/Pro-induced ERK phosphorylation. Cells were treated as in g and then stimulated with or without 1 μM NE for the indicated lengths of time. Relative phosphorylation levels of AMPK and ERK were quantified using NIH image program. Immunoblots were normalized to total loaded protein. *p<0.05 vs. WT. **p<0.05 vs. control.

### DETAILED DESCRIPTION OF THE INVENTION

We have discovered that adiponectin can be used to promote angiogenesis. Although not wishing to be bound to theory, we believe that the angiogenesis promotion is through activation of AMPK- and phosphatidylinositol-3-kinase (PI3-kinase)-AKT-dependent pathways in endothelial cells. We have also discovered that adiponectin inhibits hypertrophic signaling in cardiac myocytes and myocardium. We believe that is through activation of AMPK signaling pathway.

Angiogenesis plays a role in a wide variety of disease processes and disorders. For example, injured tissue requires angiogenesis for tissue growth and it is desirable to potentiate or promote angiogenesis in order to promote tissue healing and growth. Thus, for example, adiponectin can be used to treat patients with ischemic limbs in which there is abnormal, i.e. poor circulation as a result of diabetes, or other conditions. In addition, adiponectin can be used to treat chronic wounds which do not heal and therefore could benefit from the increase in vascular cell proliferation and neovascularization.

Adiponectin can also be used to treat a variety of cardiac disorders. As used herein, the term “cardiac disorders” includes cardiac problems of any etiology, including but not limited to, diastolic dysfunction, systolic dysfunction, cardiac hypertrophy, infectious myocarditis, inflammatory myocarditis, chemical myocarditis, cardiomyopathy of any etiology, hypertrophic cardiomyopathy, congenital cardiomyopathy, cardiomyopathy associated with ischemic heart disease or myocardial infarction and heart failure. The term “cardiac disorders”, as used herein, does not encompass arteriosclerosis. Further, as used herein, the term “cardiac disorder” is intended to encompass disorders that may or may not be associated with tissue that has a decrease in blood flow. Preferably, the cardiac disorder is cardiac hypertrophy. In another preferred embodiment, the cardiac disorder is related to decreased blood flow, for example myocardial infarction; and in that situation preferably the adiponectin is used to promote angiogenesis.

Adiponectin protein useful in the present invention can be produced in any of a variety of methods including isolation from natural sources including tissue, production by recombinant DNA expression and purification, and the like. Adiponectin protein can also be provided “in situ” by introduction of a nucleic acid cassette containing a nucleic acid (gene) encoding the protein to the tissue of interest which then expresses the protein in the tissue.

A gene encoding adiponectin protein can be prepared by a variety of methods known in the art. For example, the gene can readily be cloned using cDNA cloning methods from any tissue expressing the protein. The accession number for the human adiponectin gene transcript is NM_{—}004797 and the rat accession number is NM_{—}144744. Protein accession numbers are NP_{—}004788 and NP_{—}653345 for human and rat respectively. See also, U.S. Pat. No. 5,869,330; US20020132773; US200230147855 and US200230176328.

The nucleotide sequences of particular use in the present invention, which, encode for adiponectin protein, include various DNA segments, recombinant DNA (rDNA) molecules and vectors constructed for expression of adiponectin protein. DNA molecules (segments) of this invention therefore can comprise sequences which encode whole structural genes, fragments of structural genes encoding a protein fragment having the desired biological activity such as promoting angiogenesis, and transcription units.

A preferred DNA segment is a nucleotide sequence which encodes adiponectin protein as defined herein, or biologically active fragment thereof. By biologically active, it is meant that the expressed protein will have at least some of the biological activity of the intact protein found in a cell for the desired purpose. Preferably it has at least 50% of the activity, more preferably at least 75%, still more preferably at least 90% of the activity.

A preferred DNA segment codes for an amino acid residue sequence substantially the same as, and preferably consisting essentially of, an amino acid residue sequence or portions thereof corresponding to human adiponectin protein described herein.

A nucleic acid is any polynucleotide or nucleic acid fragment, whether it be a polyribonucleotide of polydeoxyribonucleotide, i.e., RNA or DNA, or analogs thereof such as PNA.

DNA segments are produced by a number of means including chemical synthesis methods and recombinant approaches, preferably by cloning or by polymerase chain reaction (PCR).

The adiponectin gene of this invention can be cloned from a suitable source of genomic DNA or messenger RNA (mRNA) by a variety of biochemical methods. Cloning these genes can be conducted according to the general methods known in the art. Sources of nucleic acids for cloning an adiponectin gene suitable for use in the methods of this invention can include genomic DNA or messenger RNA (mRNA) in the form of a cDNA library, from a tissue believed to express these proteins.

A preferred cloning method involves the preparation of a cDNA library using standard methods, and isolating the adiponectin-encoding or nucleotide sequence by PCR amplification using paired oligonucleotide primers based on nucleotide sequences described herein. Alternatively, the desired cDNA clones can be identified and isolated from a cDNA or genomic library by conventional nucleic acid hybridization methods using a hybridization probe based on the nucleic acid sequences described herein. Other methods of isolating and cloning suitable adiponectin-encoding nucleic acids are readily apparent to one skilled in the art.

The invention also includes a recombinant DNA molecule (rDNA) containing a DNA segment encoding adiponectin as described herein. An expressible rDNA can be produced by operatively (in frame, expressibly) linking a promoter to an adiponectin encoding DNA segment of the present invention, creating a cassette. The cassette can be administered by any known means including catheter, vector, gene gun, etc.

The choice of promoters to which a DNA segment of the present invention is operatively linked depends directly, as is well known in the art, on the functional properties desired, e.g., protein expression, and the host cell to be transformed. Promoters that express in prokaryotic and eukaryotic systems are familiar to one of ordinary skill in the art, and are described by Sambrook et al., Molecular Cloning: A Laboratory Manual Cold Spring Harbor Laboratory (2001). Preferably one uses an inducible promoter.

Expression vectors compatible with eukaryotic cells, preferably those compatible with vertebrate cells, can be used to form the recombinant DNA molecules of the present invention. Eukaryotic cell expression vectors are well known in the art and are available from several commercial sources. Typically, such vectors are provided containing convenient restriction sites for insertion of the desired DNA segment. These vectors can be viral vectors such as adenovirus, adeno-associated virus, pox virus such as an orthopox (vaccinia and attenuated vaccinia), avipox, lentivirus, murine moloney leukemia virus, etc.

Additionally, a nucleotide sequence that encodes adiponectin, or biologically active fragment thereof, can also be delivered using other means. Such gene transfer methods for gene therapy fall into three broad categories: (1) physical (e.g., electroporation, direct gene transfer and particle bombardment), (2) chemical (e.g. lipid-based carriers and other non-viral vectors) and (3) biological (e.g. virus derived vectors). For example, non-viral vectors such as liposomes coated with DNA may be directly injected intravenously into the patient. It is believed that the liposome/DNA complexes are concentrated in the liver where they deliver the DNA to macrophages and Kupffer cells.

Gene therapy methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then reimplanted in the patient. In in vitro gene transfer, the transformed cells are cells growing in culture, such as tissue culture cells, and not particular cells from a particular patient. These “laboratory cells” are transfected, the transfected cells are selected and expanded for either implantation into a patient or for other uses. In vivo gene transfer involves introducing the DNA into the cells of the patient when the cells are within the patient. All three of the broad based categories described above may be used to achieve gene transfer in vivo, ex vivo, and in vitro.

Mechanical (i.e. physical) methods of DNA delivery can be achieved by direct injection of DNA, such as catheters, preferably a catheter containing the cassette in a suitable carrier, microinjection of DNA into germ or somatic cells, pneumatically delivered DNA-coated particles, such as the gold particles used in a “gene gun,” and inorganic chemical approaches such as calcium phosphate transfection. It has been found that physical injection of plasmid DNA into muscle cells yields a high percentage of cells which are transfected and have a sustained expression of marker genes. The plasmid DNA may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylacetic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Particle-mediated gene transfer may also be employed for injecting DNA into cells, tissues and organs. With a particle bombardment device, or “gene gun,” a motive force is generated to accelerate DNA-coated high density particles (such as gold or tungsten) to a high velocity that allows penetration of the target organs, tissues or cells. Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. The techniques of particle-mediated gene transfer and electroporation are well known to those of ordinary skill in the art.

Chemical methods of gene therapy involve carrier mediated gene transfer through the use of fusogenic lipid vesicles such as liposomes or other vesicles for membrane fusion. A carrier harboring a DNA of interest can be conveniently introduced into body fluids or the bloodstream and then site specifically directed to the target organ or tissue in the body. Liposomes, for example, can be developed which are cell specific or organ specific. The foreign DNA carried by the liposome thus will be taken up by those specific cells. Injection of immunoliposomes that are targeted to a specific receptor on certain cells can be used as a convenient method of inserting the DNA into the cells bearing the receptor. Another carrier system that has been used is the asialoglycoprotein/polylysine conjugate system for carrying DNA to hepatocytes for in vivo gene transfer.

Transfected DNA may also be complexed with other kinds of carriers so that the DNA is carried to the recipient cell and then resides in the cytoplasm or in the nucleoplasm of the recipient cell. DNA can be coupled to carrier nuclear proteins in specifically engineered vesicle complexes and carried directly into the nucleus.

Carrier mediated gene transfer may also involve the use of lipid-based proteins which are not liposomes. For example, lipofectins and cytofectins are lipid-based positive ions that bind to negatively charged DNA, forming a complex that can ferry the DNA across a cell membrane. Fectins may also be used. Another method of carrier mediated gene transfer involves receptor-based endocytosis. In this method, a ligand (specific to a cell surface receptor) is made to form a complex with a gene of interest and then injected into the bloodstream; target cells that have the cell surface receptor will specifically bind the ligand and transport the ligand-DNA complex into the cell.

Biological gene therapy methodologies usually employ viral vectors to insert genes into cells. The term “vector” as used herein in the context of biological gene therapy means a carrier that can contain or associate with specific polynucleotide sequences and which functions to transport the specific polynucleotide sequences into a cell. The transfected cells may be cells derived from the patient's normal tissue, the patient's diseased tissue, or may be non-patient cells. Examples of vectors include plasmids and infective microorganisms such as viruses, or non-viral vectors such as the ligand-DNA conjugates (preferably the ligand is to a receptor preferentially expressed on the cell of interest. In one embodiment, one uses an antibody as the ligand.), liposomes, and lipid-DNA complexes discussed above.

Viral vector systems which may be utilized in the present invention include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picomavirus vectors; (i) pox virus vectors such as an orthopox, e.g., vaccinia virus vectors or avipox, e.g. canary pox or fowl pox; and (j) a helper-dependent or gutless adenovirus. In the preferred embodiment the vector is an adenovirus.

Thus, a wide variety of gene transfer/gene therapy vectors and constructs are known in the art. These vectors are readily adapted for use in the methods of the present invention. By the appropriate manipulation using recombinant DNA/molecular biology techniques to insert an operatively linked adiponectin encoding nucleic acid segment into the selected expression/delivery vector, many equivalent vectors for the practice of the present invention can be generated.

It will be appreciated by those of skill that cloned genes readily can be manipulated to alter the amino acid sequence of a protein. The cloned gene for adiponectin can be manipulated by a variety of well known techniques for in vitro mutagenesis, among others, to produce variants of the naturally occurring human protein, herein referred to as muteins, that may be used in accordance with the invention.

The variation in primary structure of muteins of adiponectin useful in the invention, for instance, may include deletions, additions and substitutions. The substitutions may be conservative or non-conservative. The differences between the natural protein and the mutein generally conserve desired properties, mitigate or eliminate undesired properties and add desired or new properties.

Similarly, techniques for making small oligopeptides and polypeptides that exhibit activity of larger proteins from which they are derived (in primary sequence) are well known and have become routine in the art. Thus, peptide analogs of proteins of the invention, such as peptide analogs of adiponectin that exhibit antagonist activity also are useful in the invention.

Mimetics also can be used in accordance with the present invention to modulate angiogenesis. The design of mimetics is known to those skilled in the art, and is generally understood to be peptides or other relatively small molecules that have an activity the same or similar to that of a larger molecule, often a protein, on which they are modeled.

Variations and modifications to the above protein and vectors can be used to increase or decrease adiponectin expression, and to provide means for targeting. For example, adiponectin can be linked with a molecular counterligand for endothelial cell adhesion molecules, such as PECAM-adiponectin, to make these agents tissue specific.

In one embodiment, the protein or fragment thereof is linked to a carrier to enhance its bioavailability. Such carriers are known in the art and include poly(alkyl) glycol such as poly ethylene glycol (PEG).

In one aspect, the present invention provides for a method for the modulation of angiogenesis in a tissue associated with a disease process or condition, and thereby affect events in the tissue which depend upon angiogenesis. Generally, the method comprises administering to the tissue, associated with, or suffering from a disease process or condition, an angiogenesis-modulating amount of a composition comprising adiponectin protein or a nucleic acid vector expressing adiponectin.

Any of a variety of tissues, or organs comprised of organized tissues, can support angiogenesis in disease conditions including heart, skin, muscle, gut, connective tissue, brain tissue, nerve cells, joints, bones and the like tissue in which blood vessels can invade upon angiogenic stimuli.

In one aspect of the invention, adiponectin is used to treat cardiac disorders.

In one preferred embodiment, the cardiac disorder is associated with myocardial tissue that has a decreased blood supply, including, but not limited to, coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangiitis obliterans, thrombotic disorders, vasculitis, myocardial infarction, and the like.

In one preferred embodiment the cardiac disorder is cardiac hypertrophy. As used herein, the term “cardiac hypertrophy” refers to the process in which adult cardiac myocytes respond to stress through hypertrophic growth.

In one preferred embodiment, the cardiac disorder is heart failure that can be due to a variety of causes, including but not limited to, congestive heart failure, heart failure with diastolic dysfunction, heart failure with systolic dysfunction, heart failure associated with cardiac hypertrophy, and heart failure that develops as a result of chemically induced cardiomyopathy, congenital cardiomyopathy, and cardiomyopathy associated with ischemic heart disease or myocardial infarction.

The preferred patient to be treated according to the present invention is a human patient, although the invention is effective with respect to all mammals.

Thus, the method embodying the present invention comprises administering to a patient a therapeutically effective amount of a physiologically tolerable composition containing adiponectin protein or nucleic acid vector for expressing adiponectin protein.

The dosage ranges for the administration of adiponectin protein depend upon the form of the protein, and its potency, as described further herein, and are amounts large enough to produce the desired effect in which angiogenesis is potentiated and the disease symptoms mediated by lack of angiogenesis are ameliorated. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication. Typically, the dosage ranges from 0.01 pg/kg body weight to 1 mg/kg body weight.

A therapeutically effective amount is an amount of adiponectin protein, or nucleic acid encoding for adiponectin, that is sufficient to produce a measurable modulation of angiogenesis in the tissue being treated, i.e., angiogenesis-modulating amount. Modulation of angiogenesis can be measured or monitored by the CAM assay, or by other methods known to one skilled in the art. Preferably, the modulation is an increase in angiogenesis.

A therapeutically effective amount of adiponectin protein, or nucleic acid encoding for adiponectin, for treatment of a particular cardiac disorder can be measured by means known to those skilled in the art. For example, a therapeutically effective amount comprises an amount able to reduce one or more symptoms of the cardiac dysfunction, such as reduced exercise capacity, reduced blood ejection volume, increased left or right ventricular end diastolic pressure, increased pulmonary capillary wedge pressure, reduced cardiac output, cardiac index, increased pulmonary artery pressures, increased left or right ventricular end systolic and diastolic dimensions, and increased left or right ventricular wall stress and wall tension.

The adiponectin protein or nucleic acid vector expressing such protein can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, compositions of the invention can be administered intravenously, intraperitoneally, intramuscularly, subcutaneously, intracavity, transdermally, and can be delivered by peristaltic means, if desired.

The therapeutic compositions containing adiponetic protein or nucleic acid vector expressing the protein can be conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

Adiponectin protein and vectors may be adapted for catheter-based delivery systems including coated balloons, slow-release drug-eluting stents, microencapsulated PEG liposomes, or nanobeads for delivery using direct mechanical intervention with or without adjunctive techniques such as ultrasound.

When treating a disorder associated with insufficient levels of angiogenesis, the adiponectin protein of the invention may be combined with a therapeutically effective amount of another pro-angiogenesis factor and/or vasculogenic agent such as, transforming growth factor alpha (TGF-α), vascular endothelial cell growth factor (VEGF), acidic and basic fibroblast growth factor (FGF), tumor necrosis factor (TNF), and platelet derived growth factor (PDGF).

In addition, the adiponectin protein of the invention may further be combined with a therapeutically effective amount another agent known to be effective at treating cardiovascular disorders.

Any diseases or condition that would benefit from the potentiation of angiogenesis can be treated by methods of the present invention. For example, stimulation of angiogenesis can aid in the enhancement of collateral circulation where there has been vascular occlusion or stenosis (e.g. to develop a “biopass” around an obstruction of an artery, vein, or of a capillary system). Specific examples of such conditions or disease include, but are not necessarily limited to, coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangiitis obliterans, thrombotic disorders, vasculitis, and the like.

Other conditions or diseases that can be prevented using the methods of the invention include, but are not necessarily limited to, heart attack (myocardial infarction) or other vascular death, stroke, death or loss of limbs associated with decreased blood flow, and the like. In addition, the methods of the invention can be used to accelerate healing of wounds or ulcers; to improve the vascularization of skin grafts or reattached limbs so as to preserve their function and viability; to improve the healing of surgical anastomoses (e.g., as in re-connecting portions of the bowel after gastrointestinal surgery); and to improve the growth of skin or hair.

In one preferred embodiment, the methods of the invention are used to treat vascular complications of diabetes.

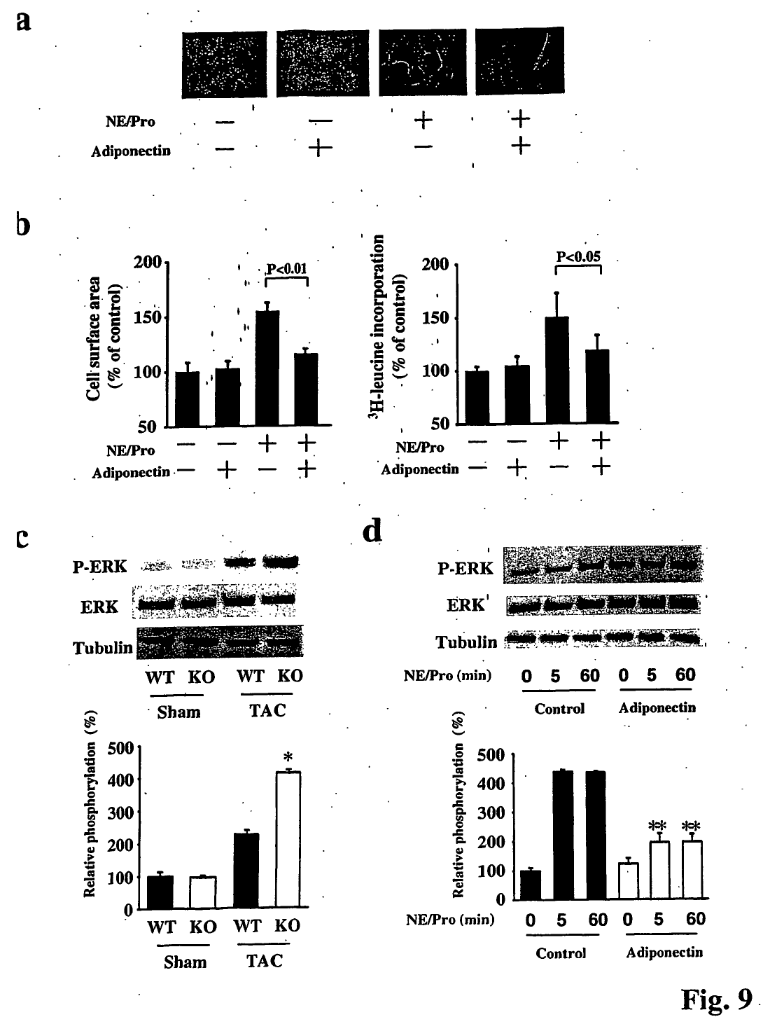

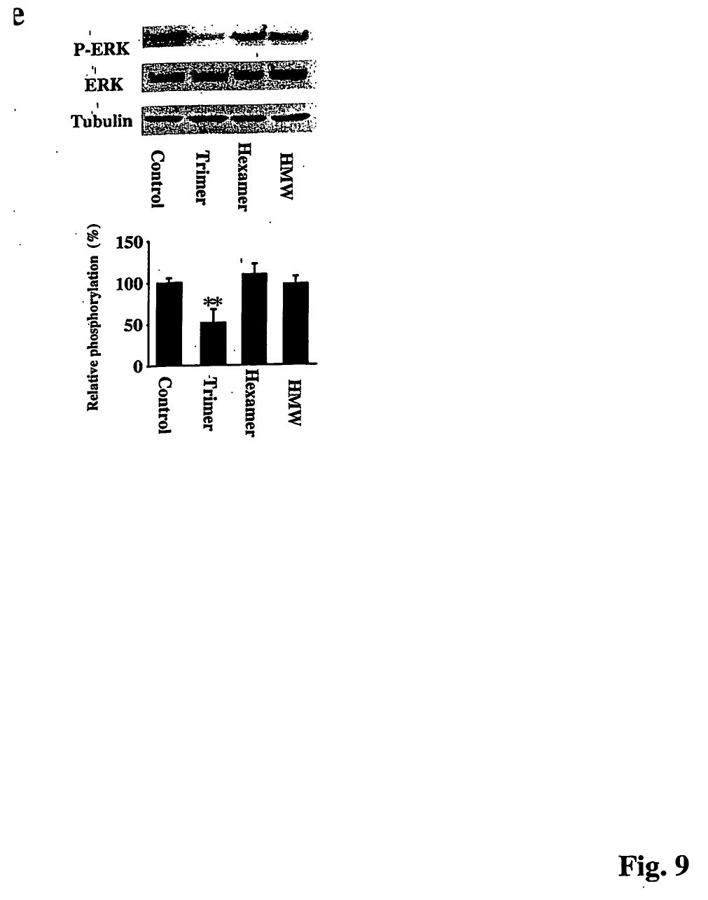

In one preferred embodiment, one uses different oligimeric forms of adiponectin for different effects. Preferably, a trimer is used to suppress a AR-stimulated ERK phosphorylation, and/or to block the increase in monocyte size. Preferably, the hexamer or MHW form is used for vascular-protective situations (See FIGS. 9A-9E).

In a one preferred embodiment, the methods of the invention are used to treat cardiac disorders associated with diabetes, such as hypertrophic cardiac myopathy.

The present invention provides therapeutic compositions useful for practicing the therapeutic methods described herein. Therapeutic compositions of the present invention contain a physiologically tolerable carrier together with adiponectin protein or vector capable of expressing adiponectin protein as described herein, dissolved or dispersed therein as an active ingredient. In a preferred embodiment, the therapeutic composition is not immunogenic when administered to a mammal or human patient for therapeutic purposes.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

For topical application, the carrier may in the form of, for example, and not by way of limitation, an ointment, cream, gel, paste, foam, aerosol, suppository, pad or gelled stick.

The amount of the active adiponectin protein (referred to as “agents”) used in the invention that will be effective in the treatment of a particular disorder or condition will depend on the nature of the disorder or condition, and can be determined by standard clinical techniques. In addition, in vitro assays such as those discussed herein may optionally be employed to help identify optimal dosage ranges.

The precise dose to be employed in the formulation will also depend on the route of administration, and the seriousness of the disease or disorder, and should be decided according to the judgment of the practitioner and each patient's circumstances. Suitable dosage ranges for administration of agents are generally about 0.01 pg/kg body weight to 1 mg/kg body weight. Effective doses may be extrapolated from dose-response curves derived from in vitro or animal model test bioassays or systems.

Administration of the doses recited above can be repeated. In a preferred embodiment, the doses recited above are administered 2 to 7 times per week. The duration of treatment depends upon the patient's clinical progress and responsiveness to therapy.

The invention also contemplates an article of manufacture which is a labeled container for providing adiponectin protein of the invention. An article of manufacture comprises packaging material and a pharmaceutical agent contained within the packaging material.

The pharmaceutical agent in an article of manufacture is any of the compositions of the present invention suitable for providing adiponectin protein and formulated into a pharmaceutically acceptable form as described herein according to the disclosed indications. Thus, the composition can comprise adiponectin protein or a DNA molecule which is capable of expressing the protein.

The article of manufacture contains an amount of pharmaceutical agent sufficient for use in treating a condition indicated herein, either in unit or multiple dosages.

The packaging material comprises a label which indicates the use of the pharmaceutical agent contained therein, e.g., for treating conditions assisted by potentiation of angiogenesis, and the like conditions disclosed herein.

The label can further include instructions for use and related information as may be required for marketing. The packaging material can include container(s) for storage of the pharmaceutical agent.

As used herein, the term packaging material refers to a material such as glass, plastic, paper, foil, and the like capable of holding within fixed means a pharmaceutical agent. Thus, for example, the packaging material can be plastic or glass vials, laminated envelopes and the like containers used to contain a pharmaceutical composition including the pharmaceutical agent.

In preferred embodiments, the packaging material includes a label that is a tangible expression describing the contents of the article of manufacture and the use of the pharmaceutical agent contained therein.

The references cited throughout this application are herein incorporated by reference.

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications and publications cited herein are incorporated herein by reference.

### EXAMPLE 1

Materials

Phospho-AMPK (Thr172), pan-α-AMPK and phospho-Akt (Ser473), phospho-eNOS (Ser1177) phospho-p42/44 extracellular signal-regulated kinase (ERK) (Thr 202/Tyr 204), ERK, and Akt antibodies were purchased from Cell Signaling Technology (Beverly, Mass.). c-Myc tag antibody was purchased from Upstate biotechnology (Lake Placid, N.Y.). eNOS antibody was purchased from Santa Cruz Biotechnology (Santa Cruz, Calif.). Tubulin antibody was purchased from Oncogene (Cambridge, Mass.). Recombinant human VEGF was purchased from Sigma (St. Louis, Mo.).

Recombinant Proteins

Mouse adiponectin (amid acids 15-247) was cloned into the bacterial expression vector pTrcHisB (Amersham Pharmacia Biotech, Piscataway, N.J.). The histidine-tagged proteins were purified using nickel-ion agarose column, monoQ column, and, for removal of lipopolysaccharide, Detoxi-Gel Affinity Pak column (Pierce, Rockford, Ill.).

Cell Culture Adenoviral Infection and Western Blot Analysis.

Human umbilical vein endothelium cells (HUVECs) were cultured in endothelial cell growth medium-2 (EGM-2, San Diego, Calif.). Before each experiment, cells were placed in endothelial cell basal medium-2 (EBM-2, San Diego, Calif.) with 0.5% fetal bovine serum (FBS) for 16 h for serum-starvation. Experiments were performed by the addition of the indicated amount of mouse recombinant adiponectin, VEGF or vehicle for the indicated lengths of time. In some experiments, HUVECs were infected with adenoviral constructs encoding dominant-negative AMPKα2²⁸, dominant-negative AKT1¹⁹ or green fluorescence protein (GFP) at a multiplicity of infection (MOI) of 50 for 24 h. In some experiments, HUVECs were pretreated with LY294002 (10 μM) or vehicle for 1 h before stimulation with adiponectin. Cell lysates were resolved by SDS-PAGE. The membranes were immunoblotted with the indicated antibodies at a 1:1000 dilution followed by the secondary antibody conjugated with horseradish peroxidase (HRP) at a 1:5000 dilution. ECL-PLUS Western Blotting Detection kit (Amersham Pharmacia Biotech, Piscataway, N.J.) was used for detection.

Migration Assay

Migration activity was measured using a modified Boyden chamber assay. Serum-starved cells were trypsinized and resuspended in EGM-2 with 0.5% FBS. Cell suspension (250 μl, 2.0×10⁴ cells/well) were added to the transwell fibronectin-coated insert (6.4 mm diameter, 3.0 μm pore size, Becton Dickinson, Franklin Lakes, N.J.). Then 750 μl of EGM-2 with 0.5% FBS supplemented with adiponectin (30 μg/ml), VEGF (20 ng/ml) or bovine serum albumin (BSA) (30 μg/ml) were added to lower chamber and incubated for 4 h. Migrated cells on the lower surface of the membrane were fixed, stained with Giemsa stain solution and eight random microscopic fields per well were quantified. All assays were performed in triplicate.

Tube Formation Assay

The formation of vascular-like structures by HUVECs on growth factor-reduced Matrigel (Becton Dickinson) was performed as previously described²⁸. Twenty-four-well culture plates were coated with Matrigel according to the manufacturer's instructions. Serum-starved HUVECs were seeded on coated plates at 5×10⁴ cells/well in EGM-2 with 0.5% FBS containing indicated concentrations of adiponectin, VEGF (20 ng/ml) or BSA (30 μg/ml) and incubated at 37° C. for 18 h. Tube formation was observe using an inverted phase contrast microscope (Nikon, Tokyo, Japan). Images were captured with a video graphic system (DEI-750 CE Digital Output Camera, Optronics, Goleta, Calif.). The degree of tube formation was quantified by measuring the length of tubes in 3 randomly chosen fields from each well using the angiogenic activity quantification program (Kurabo, Osaka, Japan). Each experiment was repeated for 3 times.

Mouse Angiogenesis Assay

The formation of new vessels in vivo was evaluated by Matrigel plug assay as described previously²⁸. For these experiments, 400 μl of Matrigel containing adiponectin (100 μg/ml) or vehicle was injected subcutaneously into the abdomen of C57BL mice. Mice were sacrificed 14 days after the injection. The Matrigel plugs with adjacent subcutaneous tissues were carefully recovered by en bloc resection, fixed in 4% paraformaldehyde, dehydrated with 30% sucrose, and embedded in OCT compound (GTI Microsystems, Tempe, Ariz.) in liquid nitrogen. Immunohistostaining for CD31 (PECAM-1: Becton Dickinson) were performed on adjacent frozen sections. Primary antibody was used at a 1:50 dilution followed by incubation of secondary antibody (HRP-conjugated anti-rat IgG at a 1:100 dilution). The AEC Substrate Pack (Biogenex, San Ramon, Calif.) was used for detection. CD31-positive capillaries were counted in 4 randomly chosen low-power (×100) microscopic fields.

Rabbit Corneal Angiogenesis Assay

Rabbit corneal assay was performed with minor modification as previously described³³. Male New Zealand white rabbits weighing 3.0-3.9 kg were used. Two pockets, about 2×3 mm size and 5 mm apart, were surgically prepared in the cornea extending toward a point 2 mm from the limbus. Hydron pellets, which contain indicated amount of adiponectin, VEGF (100 ng) or PBS and enables the slow release of it³⁴, were implanted into the pocket. On day 7 after surgery, eyes were photographed and cornea neovascularization was examined in a single blind manner. The angiogenic activity was evaluated on the basis of the number and growth rate of newly formed capillaries. An angiogenic score was calculated (vessel density×distance from limbus)³². A density value of 1 corresponded to 0-25 vessels per cornea, 2 from 25-30, 3 from 50-75, 4 from 75-100 and 5 for >100 vessels.

Statistic Analysis

Data are presented as mean±SE. Differences were analyzed by Student's unpaired t test. A level of P<0.05 was accepted as statistically significant.

Results

Adiponectin Accelerates Vascular Structure Formation In Vitro

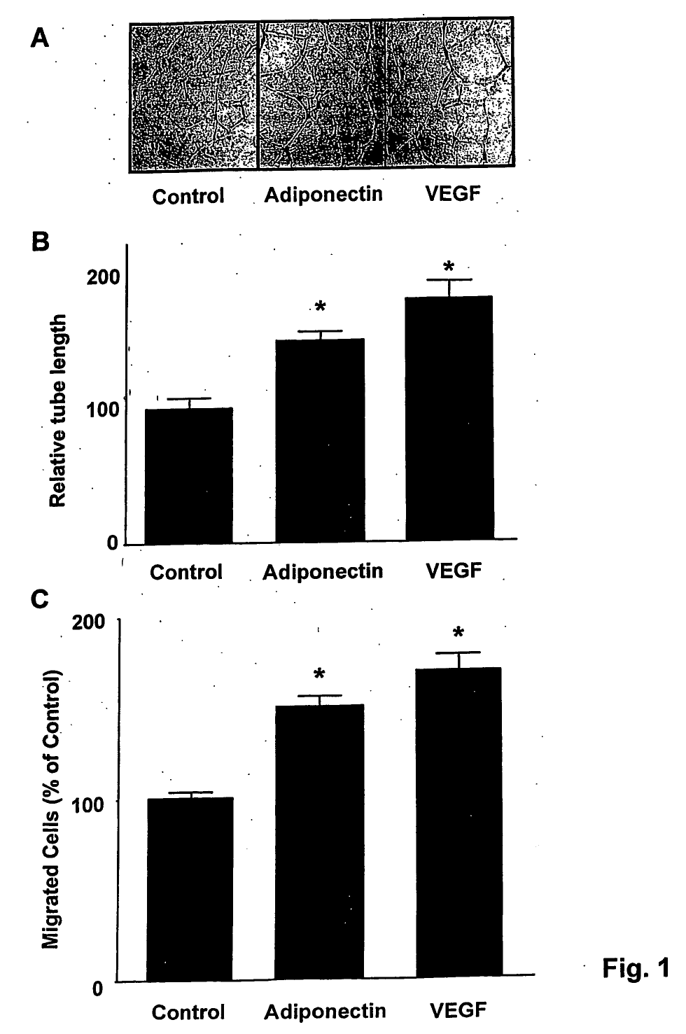

We first examined whether adiponectin affected endothelial cell differentiation into capillary-like structure when HUVECs were plated on a Matrigel matrix. Treatment with a physiological concentration of adiponectin promoted the formation of capillary-like tubes in a manner similar to VEGF (FIG. 1A). Quantitative analyses of tube structure length revealed a trend toward increased tube length in the VEGF-treated cultures relative to adiponectin, but this was not statistically significant (FIG. 1B). To test whether adiponectin modulated the endothelial migration, a modified Boyden chamber assay was performed. Adiponectin significantly stimulated HUVEC migration, as did VEGF (FIG. 1C). Quantitative analyses revealed a trend toward greater migration with VEGF compared to adiponectin, but this was not statistically significant. Adiponectin also induced the endothelial migration in a cell-wounding assay (N. Ouchi et al., unpublished data). These result suggest that adiponectin promotes pro-angiogenic cellular responses in endothelial cells.

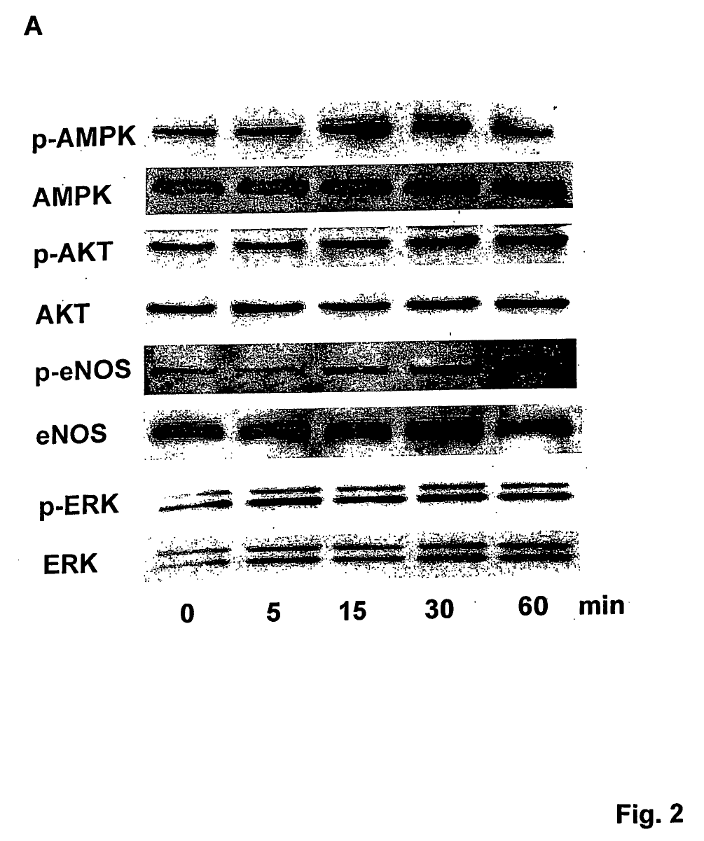

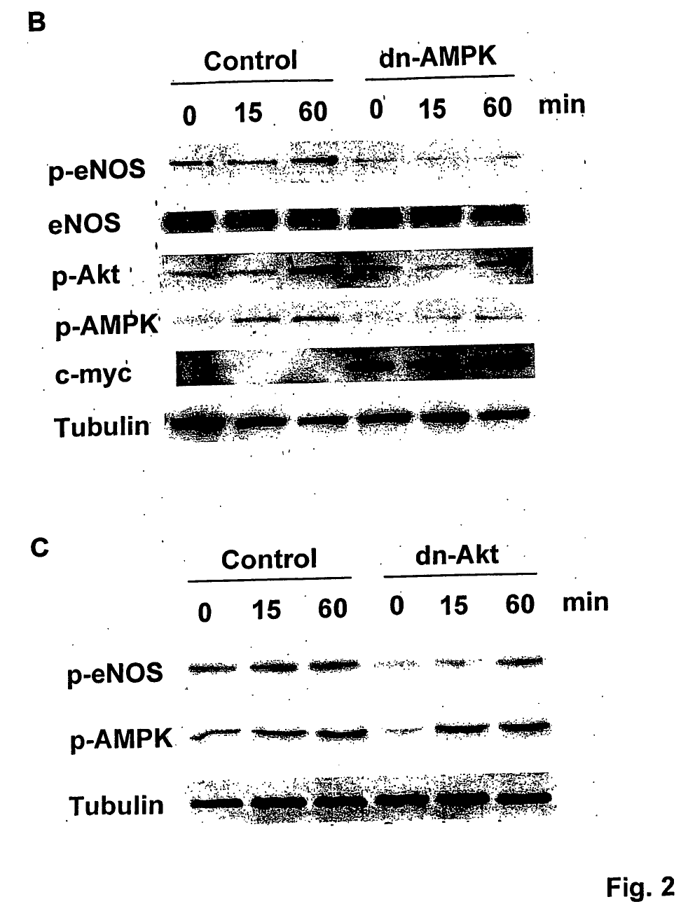

Adiponectin induces the phosphorylation of AMPK, Akt and eNOS Endothelial AMPK signaling is associated with the regulation of angiogenesis under certain conditions²⁸. Therefore, to test whether adiponectin induces AMPK signaling in endothelial cells, cultured HUVECs were incubated with adiponectin, and AMPK phosphorylation at Thr 172 of α subunit was assessed by Western blot analyses. Treatment of HUVECs with adiponectin enhanced the phosphorylation of AMPK in a time-dependent manner with maximal AMPK phosphorylation occurring at 15 minutes (FIG. 2A). Akt plays important roles in the angiogenic response to several growth factors and cytokines¹⁸. Therefore, the effect of adiponectin on the activating phosphorylation of Akt at Ser 473 was investigated. Adiponectin treatment led to a time-dependent increase in Akt phosphorylation (FIG. 2A). In contrast to these signaling protein kinases, adiponectin treatment had no effect on the phosphorylation of ERK at Thr 202/Tyr 204 (FIG. 2A). Both AMPK and Akt can phosphorylate eNOS at Ser 1179^{22,23,35,36}. Therefore, eNOS phosphorylation was, examined in these cultures. Adiponectin stimulation promoted a time-dependent increase in eNOS phosphorylation at Ser 1179, but had no effect on eNOS protein levels (FIG. 2A).

The regulation of eNOS by mitogen-stimulated phosphorylation is complicated by the possibility of AMPK-Akt cross-talk^{28,37}. To examine the relative contribution of AMPK and Akt to the regulation of adiponectin-induced phosphorylation of eNOS, HUVECs were transduced either with an adenoviral vector expressing a c-Myc-tagged dominant-negative mutant of AMPK (ad-dnAMPK) or dominant-negative Akt (ad-dnAkt). Transduction with ad dnAMPK suppressed adiponectin-induced AMPK and eNOS phosphorylation (FIG. 2B). Transduction with ad-dnAMPK also blocked adiponectin-induced phosphorylation of Akt suggesting signaling cross-talk between these two protein kinases (FIG. 2B). Of note, transduction with ad-dnAkt suppressed the adiponectin-induced phosphorylation of eNOS without altering that of AMPK (FIG. 2C). These data indicated that Akt is a downstream kinase of AMPK and that Akt mediates eNOS phosphorylation downstream from adiponectin/AMPK.

AMPK and Akt Signaling are Required for Adiponectin-Stimulated Migration and Differentiation

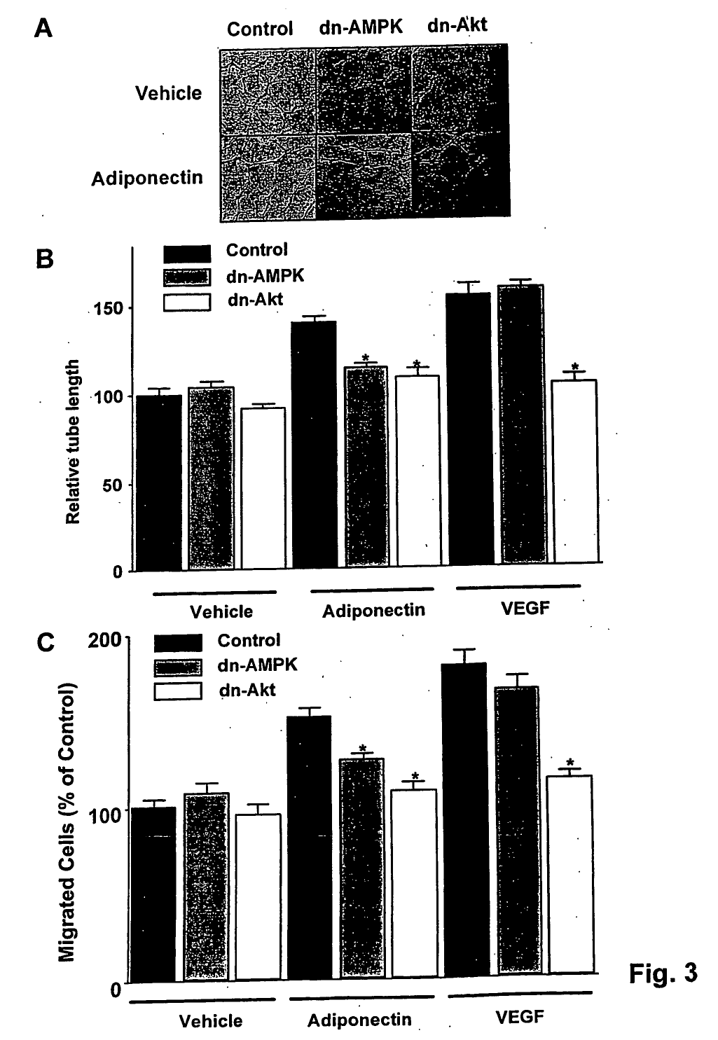

To test whether AMPK and Akt signaling participate in adiponectin-stimulated endothelial differentiation and migration, HUVECs were infected with ad-dnAMPK or ad-dnAkt and evaluated in tube formation and Boyden chamber assays, respectively. Transduction with either ad-dnAMPK or ad-dnAkt suppressed adiponectin-induced endothelial tube structure formation to basal levels (FIGS. 3, A and B). In contrast, VEGF-stimulated differentiation was blocked by transduction with ad-dnAkt, but not by transduction with ad-dnAMPK (FIG. 3B). Transduction with ad-dnAMPK and ad-dnAkt had no effect on non-stimulated, basal tube formation (FIG. 3B). Adiponectin-stimulated endothelial migration was also significantly suppressed by transduction with either ad-dnAMPK or ad-dnAkt (FIG. 3C). In contrast, transduction with ad-dnAkt blocked VEGF-stimulated migration, while transduction with ad-dnAMPK had no effect (FIG. 3C). Transduction with ad-dnAMPK and ad-dnAkt had no effect on the basal migration rate (FIG. 3C). These results indicated that both AMPK and Akt signals are required for adiponectin-induced endothelial migration and differentiation, whereas only Akt signaling participates in these endothelial cell responses to VEGF.

Role of PI3-Kinase Signaling in Adiponectin-Induced Angiogenic Response

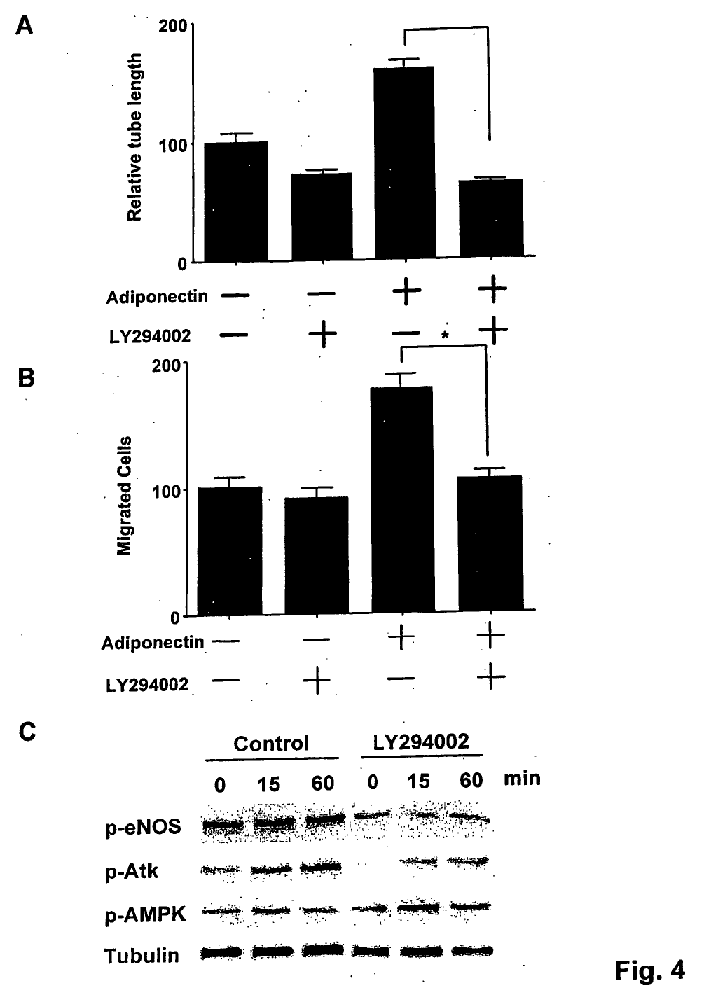

Akt is activated by many growth factors and cytokines in a PI3-kinase-dependent manner¹⁸. To investigate whether PI3-kinase signal is involved in adiponectin-induced angiogenic signaling pathway, HUVECs were incubated with PI3-kinase inhibitor, LY294002 in the absence or presence of adiponectin. Brief treatment with LY294002 abolished adiponectin-stimulated tube formation and migration (FIGS. 4, A and B). Adiponectin-stimulated the phosphorylation of Akt and eNOS was blocked by treatment with LY294002, while LY294002 treatment had no effect on AMPK phosphorylation (FIG. 4C). These data indicate that PI3-kinase is a critical for adiponectin-induced angiogenic cell responses and that PI3-kinase functions upstream from the Akt-eNOS regulatory axis in adiponectin-stimulated endothelial cells.

Adiponectin Promotes Vessel Growth In Vivo

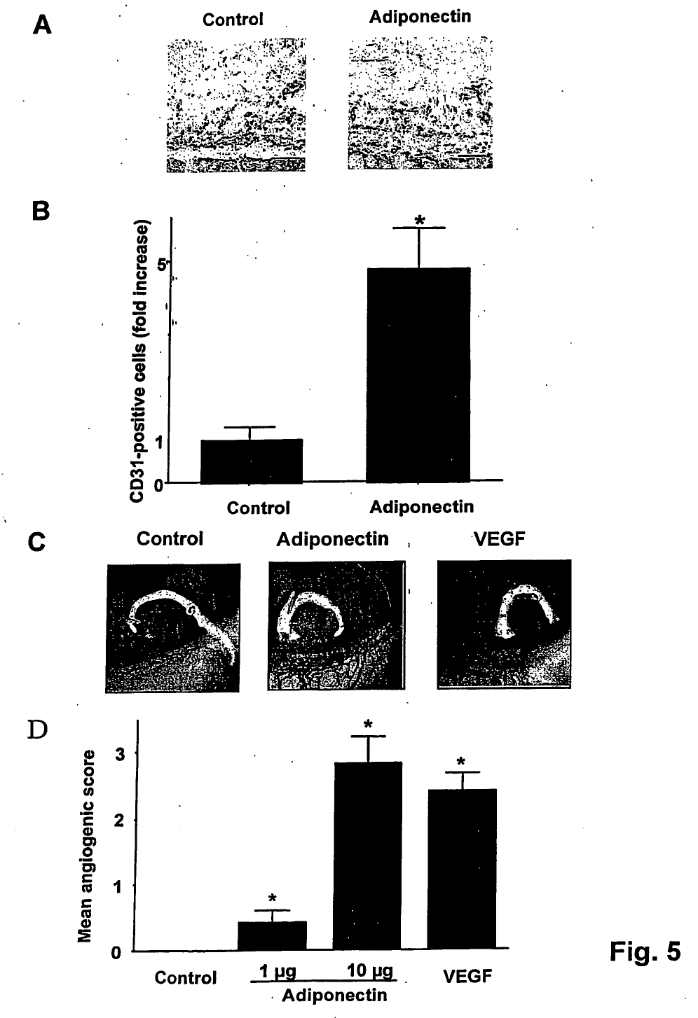

To examine the in vivo effect of adiponectin on angiogenesis, mouse Matrigel plugs and rabbit corneal assays were performed. In the Matrigel plugs assay, endothelial cell infiltration of the plugs was assessed by immunohistochemical analysis of CD31-positive cells (FIG. 5A). Quantitative analyses of histological sections revealed that plugs containing adiponectin displayed a significantly higher density of CD31-positive cells compared with controls (FIG. 5B). In addition, the angiogenic activity of adiponectin was essential in a rabbit corneal assay. Neovascularization in corneal implants containing adiponectin was markedly accelerated compared with controls (FIGS. 5, C and D). The stimulatory effect of adiponectin was comparable with that of VEGF in this model (FIGS. 5, C and D). These data show that adiponectin can promote neovascularization in vivo.

Discussion

This study shows the promotion of blood vessel growth as a new role for the adipocytokine adiponectin. Proangiogenic activity was demonstrated in two well-established models of angiogenesis, the mouse Matrigel plug and rabbit corneal assays. The ability of adiponectin to stimulate angiogenesis is likely due, at least in part, to its ability to promote endothelial cell migration and stimulate the differentiation of these cells into capillary-like structures.

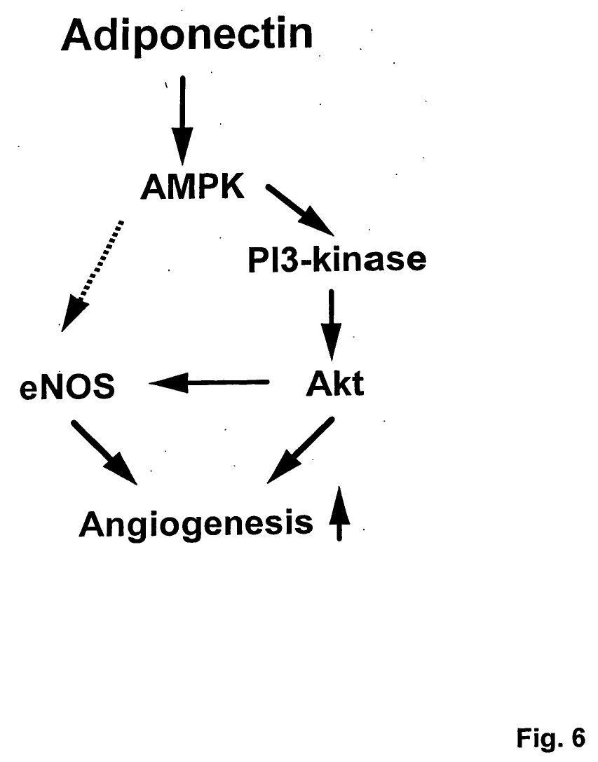

Adiponectin functions as an AMPK activator in multiple cell types^{29-32,38}. Recently, we reported that endothelial AMPK signaling is essential for angiogenesis under conditions of hypoxia, but dispensable in normoxic cells. Here it is shown that AMPK activation by adiponectin can activate angiogenic cellular responses in normoxic endothelial cells. Furthermore, it is shown that cross-talk between AMPK and Akt protein kinases results in several cellular responses downstream of adiponectin including the activating phosphorylation of eNOS at Ser 1179. Several recent reports have demonstrated the importance of AMPK-Akt cross-talk^{28,37}. While both Akt and AMPK are reported to directly phosphorylate eNOS^{22,23,35,36}, our study found that transduction with either ad-dnAMPK or ad-dnAkt effectively blocked adiponectin-induced eNOS phosphorylation. Both of these reagents also suppressed adiponectin-stimulated endothelial cell migration and differentiation. Furthermore, inhibition of AMPK signaling suppressed adiponectin-induced Akt phosphorylation, suggesting that Akt functions downstream of AMPK in adiponectin-stimulated endothelial cells (FIG. 6). Importantly, the PI3-kinase inhibitor LY294002 blocked adiponectin-stimulated cell migration, differentiation and Akt and eNOS phosphorylation, without altering the phosphorylation status of AMPK. These data indicate that the pro-angiogenic effects of adiponectin-stimulated AMPK activity are due, in part, to an activation of Akt signaling under these conditions. Although we cannot exclude the possibility that AMPK directly phosphorylates eNOS, the data is most consistent with a model that comprises an adiponectin-AMPK-PI3-kinase-Akt-eNOS signaling axis under the conditions of our assays (FIG. 6).

The hypothesis that AMPK functions upstream of Akt signaling is consistent with data obtained from studies in other systems. For example, it has been shown that the AMPK stimulator 5-aminoimidazole-4-carboxamide riboside enhances insulin-stimulated activation of IRS-1-associated PI3-kinase in C2C12 myocytes³⁹. Furthermore, adiponectin-deficient mice exhibit severe diet-induced insulin resistance that coincides with a reduction of muscle IRS-1-associated PI3-kinase activity¹⁴. Conversely, adiponectin stimulates IRS-1-associated PI3-kinase activity in C2C12 myocytes 14, and adiponectin treatment increases insulin-stimulated Akt phosphorylation in the skeletal muscle of adiponectin-treated lipoatrophic mice⁴⁰

Plasma adiponectin levels are low in patients with type 2 diabetes⁹. Low levels of adiponectin expression have also been observed in the visceral fat of diabetic fa/fa Zucker rats in comparison with lean rats⁴¹. Clinically, collateral vessel development is impaired in diabetic patients including those with myocardial and limb ischemia^{42,43} and, in animal models, there is an impaired angiogenic response following ischemic injury in nonobese diabetic mice and obese diabetic fa/fa Zucker rats^{44,45}. Therefore, low adiponectin levels may contribute the impaired collateral growth in diabetic states. Taken together, these data suggest that the exogenous supplementation of adiponectin is useful treatment for vascular complications of diabetes and other ischemic diseases.

### EXAMPLE 2

Materials

Phospho-AMPK (Thr172), pan-α-AMPK and phospho-p42/44 extracellular signal-regulated kinase (ERK) (Thr 202/Tyr 204) and total ERK antibodies and U0126 were purchased from Cell Signaling Technology (Beverly, Mass.). Tubulin antibody was from Oncogene (Cambridge, Mass.). Phospho-Acetyl CoA Carboxylase (ACC) (Ser-79), ACC and c-Myc tag antibody were purchased from Upstate biotechnology (Lake Placid, N.Y.). L-norepinephrine, DL-propranolol and Angiotensin II (AngII) were purchased from Sigma (St. Louis, Mo.). Recombinant mouse adiponectin was prepared as described previously⁶⁶. Adenovirus vectors containing the gene for β-galactosidase (Ad-βgal), full-length mouse adiponectin (Ad-APN), and dominant-negative AMPKα2 (Ad-dnAMPK) were prepared as described previously^{59,28}. The trimer, hexamer and HMW forms of adiponectin were prepared as described previously⁶³.

Transverse Aortic Constriction

Adiponectin knockout (APN-KO), wild-type (WT) and db/db mice in a C57/BL6 background were used for this study⁵⁹. Study protocols were approved by the Institutional Animal Care and Use Committee in Boston University. Mice, at the ages of 7 to 11 weeks, were anesthetized with sodium pentobarbital (50 mg/kg intraperitoneally). The chest was opened, and following blunt dissection through the intercostal muscles, the thoracic aorta was identified. A 7-0 silk suture was placed around the transverse aorta and tied around a 26-gauge blunt needle, which was subsequently removed⁷⁶. Sham-operated mice underwent a similar surgical procedure without constriction of the aorta. After 7 days, surviving mice were subjected to transthoracic echocardiography and cardiac catheterization to determine heart rate and proximal aortic pressure. Animals were then euthanized and the hearts were dissected out and weighed.

Adenovirus-Mediated Gene Transfer

The 2×10⁸ plaque-forming units of Ad-APN or Ad-β-galactosidase (βgal) were injected into the jugular vein of mice 3 days prior to the transverse aortic constriction (TAC). Echocardiography was performed at 3 days post-surgery. Mouse adiponectin levels were determined by ELISA kit (Otsuka Pharmaceutical Co. Ltd., Tokyo, Japan). The oligomeric state of adiponectin was analyzed by gel filtration chromatography as described previously⁶³.

AngII Infusion

AngII (3.2 mg/kg/day) was subcutaneously infused into APN-KO and WT mice with an implanted osmotic minipump (Alzet Co). Some mice were transduced with 2×10⁸ plaque-forming units of Ad-APN or Ad-βgal injected into the jugular vein. After 14 days, mice were subjected to transthoracic echocardiography and cardiac catheterization to determine heart rate and blood pressure.

Echocardiography

To measure left ventricular (LV) wall thickness and chamber dimensions, echocardiography was performed with an Acuson Sequoia C-256 machine using a 15-Mhz probe. After a good quality 2 dimensional image was obtained, M-mode images of the left ventricular posterior wall thickness were measured. Cardiac output was calculated by the cubed method (1.047×(LVEDD³−LVESD³)×HR).

Cell Culture and Adenoviral Infection

Primary cultures of the neonatal rat ventricular myocytes were prepared as described previously⁷⁴. The isolated myocytes were cultured in DMEM containing 7% fetal calf serum. Before each experiment, cells were placed in serum-free DMEM for 24 hours. For the adiponectin stimulation studies, 30 μg/ml of mouse recombinant adiponectin was treated for the indicated lengths of time. Experiments for norepinephrine stimulation were performed by treating cells with 30 μg/ml of mouse recombinant adiponectin or vehicle for 30 minutes. Cells were then treated with 2 μM of propranolol for 30 minutes and stimulated with 1 μM norepinephrine for the indicated lengths of time. In some experiments, the cells were infected with Ad-βgal and Ad-dnAMPK at a multiplicity of infection of 50 for 24 hours prior to treatments. Myocyte surface area was assessed using semi-automatic computer-assisted planimetry (Bioquant) from two-dimensional images of unstained cells. [³H] leucine incorporation was determined as previously described 74.

Immunohistochemical Analysis

For histological analysis, the mice were sacrificed and LV tissue was obtained at 7 days after TAC. Tissue was embedded in OCT compound (Miles, Elkhart, Ind.) and snap-frozen in liquid nitrogen. Tissue slices (5 μm in thickness) were prepared. Tissue sections were stained with hematoxylin and eosin or with Masson trichrome. The myocyte cross sectional area was calculated by measuring 200 cells per section. To determine sarcomeric F-actin organization, cultured myocytes were stained with FITC-conjugated phalloidin (Sigma, St. Louis, Mo.).

Western Blot Analysis

Heart tissue samples obtained at day 7 post-surgery were homogenized in lysis buffer containing 20 mM Tris-HCl (pH 8.0), 1% NP-40, 150 mM NaCl, 0.5% deoxycholic acid, 1 mM sodium orthovanadate, and protease inhibitor cocktail (Sigma, St. Louis, Mo.). The rat myocytes were homogenized in the same lysis buffer. The same amount of protein (50 μg) was separated with denaturing SDS 10% polyacrylamide gels. Following transfer to membranes, immunoblot analysis was performed with the indicated antibodies at a 1:1000 dilution. This was followed by incubation with secondary antibody conjugated with horseradish peroxidase at a 1:5000 dilution. ECL Western Blotting Detection kit (Amersham Pharmacia Biotech, Piscataway, N.J.) was used for detection.

Statistical Analysis

Data are presented as mean±SE. Statistical analysis was performed by analysis of variance (ANOVA), student t test, Scheffe's F test and χ² analysis. A value of P<0.05 was accepted as statistically significant.

Summary

We show that pressure overload in adiponectin-deficient mice results in increased mortality and enhanced concentric cardiac hypertrophy that is associated with increased extracellular signal-regulated kinase (ERK) and diminished AMP-activated protein kinase (AMPK) signaling in the myocardium. In our study, Adenovirus-mediated supplement of adiponectin attenuated cardiac hypertrophy in response to pressure overload in adiponectin-deficient, wild-type and diabetic db/db mice. In cardiac myocytes in vitro, adiponectin activated AMPK and inhibited agonist-stimulated hypertrophy and ERK activation. These effects were reversed by transduction with dominant-negative AMPK indicating that adiponectin inhibits hypertrophic signaling in the myocardium through activation of AMPK signaling. Thus, the use of Adiponectin represents a means for treating hypertrophic cardiomyopathy associated with diabetes and other obesity-related diseases.

Results

Role of Adiponectin in Regulating Cardiac Hypertrophy

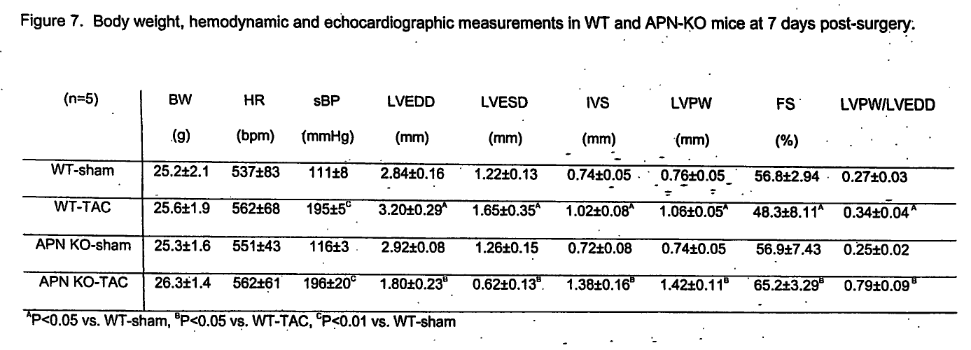

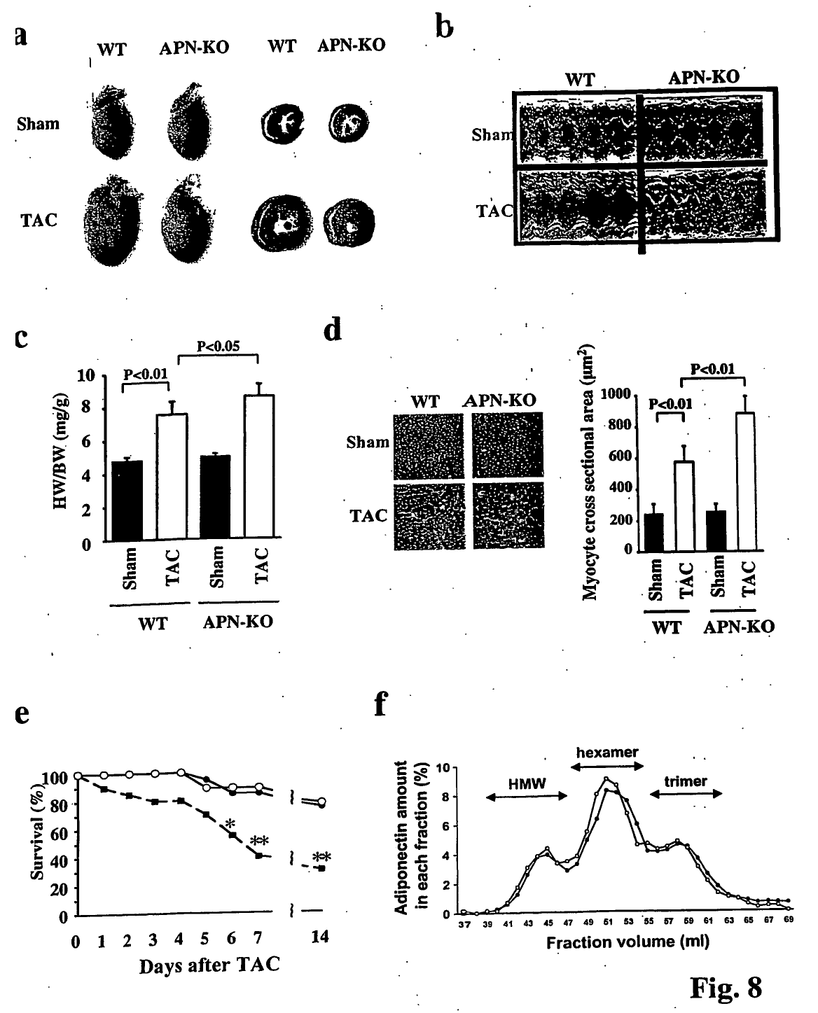

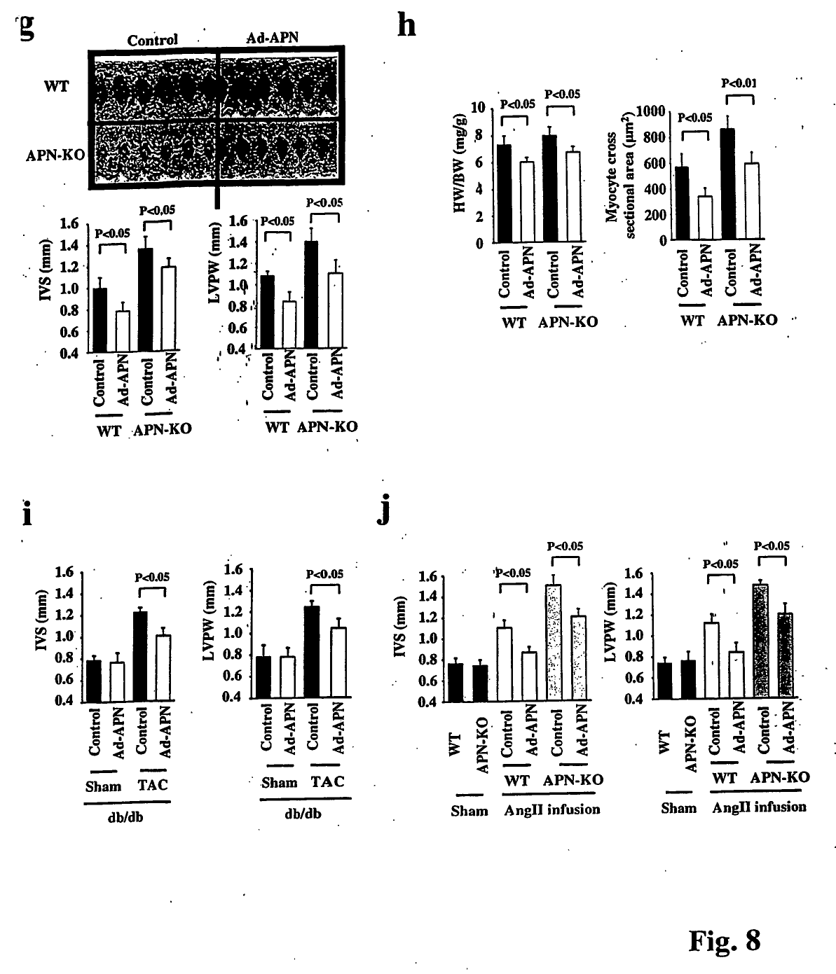

Adiponectin knockout (APN-KO) mice were subjected to pressure overload caused by transverse aortic constriction (TAC). There were no significant differences in body weight (BW) or heart rate (HR) between APN-KO mice and wild type (WT) animals after sham operation or TAC, and the increase in systolic blood pressure (sBP) after TAC was similar in WT and APN-KO mice (FIG. 7). By gross morphologic examination 7 days after TAC, APN-KO mice (as compared to WT mice) had increased left ventricular (LV) wall thickness typical of exaggerated concentric hypertrophy (FIG. 8a). Echocardiographic measurements 7 days after TAC showed decreased LV end-diastolic dimension (LVEDD) and increased interventricular septum (IVS) and LV posterior wall thickness (LVPW) in APN-KO mice, as compared to WT animals (FIG. 8b and FIG. 7). The LVPW/LVEDD ratio increased markedly in APN-KO compared to WT mice after TAC (Table 1). After TAC, heart weight (HW)/BW ratio was also increased in APN-KO mice compared to WT animals (FIG. 8c), as was myocyte cross-sectional area (FIG. 8d). The finding of markedly increased LVPW/LVEDD ratio in the setting of increased heart weight is indicative of severe concentric hypertrophy. The calculated cardiac output was 14.1±2.0, 16.2±2.6, 14.0±1.3 and 4.2±0.4 ml/min in WT/sham, WT/TAC, APN-KO/sham and APN-KO/TAC, respectively. Mortality at 6, 7 and 14 days after TAC was significantly higher in APN-KO compared to WT mice (FIG. 8e). This increased mortality in APN-KO mice could result from the dramatic decrease in cardiac output following TAC.

To confirm that the exaggerated hypertrophic response to pressure overload was due to adiponectin deficiency, APN-KO and WT mice were treated with an adenoviral vector, expressing adiponectin (Ad-APN) or a control (Ad-βgal), delivered via the jugular vein 3 days before TAC. At the time of surgery, adiponectin levels were 9.93±2.41 μg/ml in WT/control, 18.80±2.28 μg/ml in WT/Ad-APN, <0.05 μg/ml in APN-KO/control and 11.10±11.75 in APN-KO/Ad-APN. Adiponectin is present in serum as a trimer, hexamer, or high molecular weight (HMW) forms⁵³. The oligomer distribution of adenovirus-encoded adiponectin in the sera of APN-KO mice was similar to that of endogenous adiponectin in WT mice as determined by gel filtration analysis (FIG. 8f). Ad-APN treatment attenuated the TAC-induced changes in LV morphology (decreased LVEDD and increased IVS, LVPW) observed in the APN-KO mouse (FIG. 8g). Ad-APN also decreased HW/BW ratio, myocyte cross-sectional area and mortality in this model (FIG. 8 e, FIG. 8h). Collectively, these data indicate that adiponectin deficiency causes an enhanced hypertrophic response to pressure overload and is associated with increased mortality. Ad-APN treatment also attenuated the increased IVS and LVPW response to TAC in db/db mice, a model of obesity and diabetes (FIG. 8i). Finally, APN-KO mice subjected to Angiotensin II (AngII) infusion exhibited increased IVS and LVPW compared to WT mice (FIG. 8j). The increase in sBP after AngII infusion was similar in WT and APN-KO mice (130.8±5.4 mmHg in WT vs. 134.4±6.8 mmHg in APN-KO mice). Ad-APN treatment attenuated the AngII-induced changes in LV morphology observed in both the APN-KO and WT mice (FIG. 8j).

Effect of Adiponectin in Cardiac Myocytes

The effects of adiponectin in cardiac myocytes at the cellular level were shown using ventricular myocytes obtained from rats subjected to α-adrenergic receptor (αAR) stimulation with norepinephrine (NE) in the presence of propranolol (Pro)⁶¹, with or without the addition of recombinant adiponectin protein. αAR stimulation for 48 hours caused an increase in myocyte size and protein synthesis (FIG. 9a and FIG. 9b) that was associated with re-organization of sarcomeric actin (FIG. 9a), and these effects were prevented by pretreatment with adiponectin. Adiponectin alone had no effect on myocyte size, protein synthesis or actin organization. Adiponectin treatment also suppressed AngII-stimulated in myocyte hypertrophy (data not shown).

Gq-dependent activation of extracellular signal-regulated kinase (ERK) is an important mediator of myocyte hypertrophy in response to pressure overload⁶¹ and αAR stimulation⁶¹. Therefore, the effect of adiponectin on ERK phosphorylation at Thr 202/Tyr 204 was investigated by western blotting. In vivo, ERK phosphorylation was similar in myocardium from sham-operated APN-KO and WT mice, whereas pressure overload-induced ERK phosphorylation was enhanced in APN-KO compared to WT mice (FIG. 9c). In cultured cardiac myocytes, αAR stimulation induced ERK phosphorylation that was suppressed by pretreatment with adiponectin (FIG. 9d). Under the conditions of these assays, treatment with the ERK inhibitor U0126 reduced αAR-induced hypertrophy by 82.1±7.8% (p<0.01 vs. control), indicating that ERK inhibition by adiponectin contributes to the suppression of cardiac myocyte hypertrophy. Adiponectin treatment alone had no effect on ERK phosphorylation in cardiac myocytes. Adiponectin treatment also suppressed AngII-stimulated ERK phosphorylation (data not shown). The trimer form specifically suppressed αAR-stimulated ERK phosphorylation, while the hexamer or HMW forms of adiponectin had little effect (FIG. 9e). The trimer form of adiponectin also blocked the increase in myocyte size caused by αAR stimulation (data not shown). In contrast, the HMW form of adiponectin appears to be specific for the vascular-protective actions⁶³.

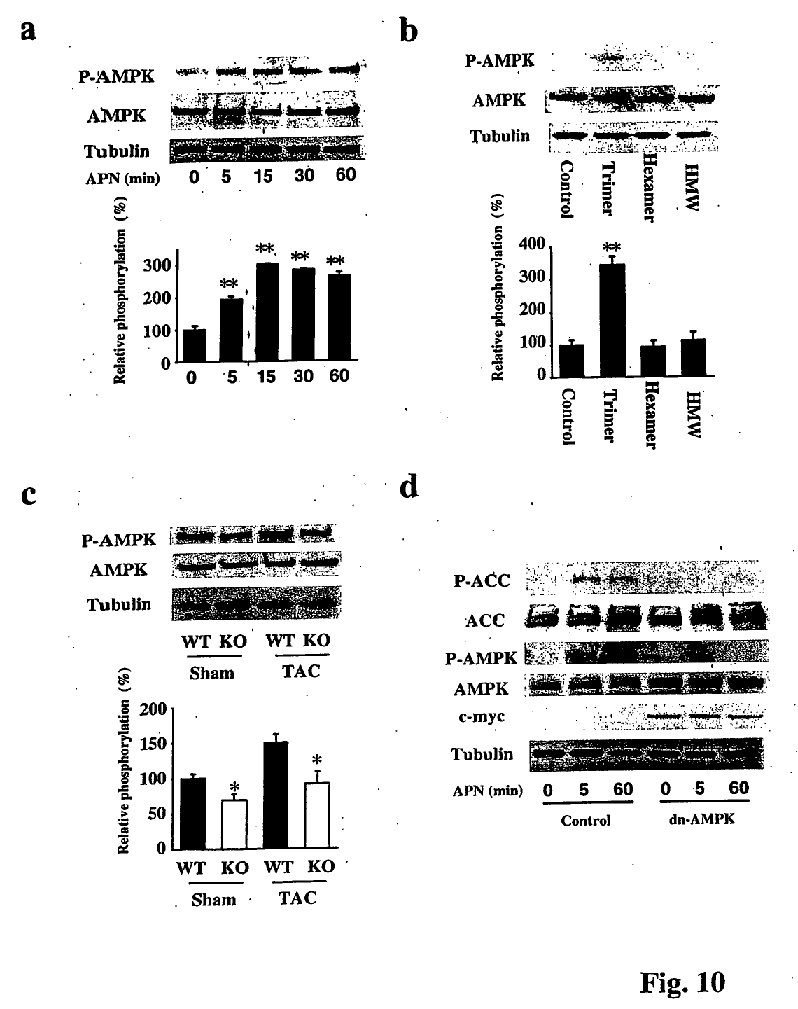

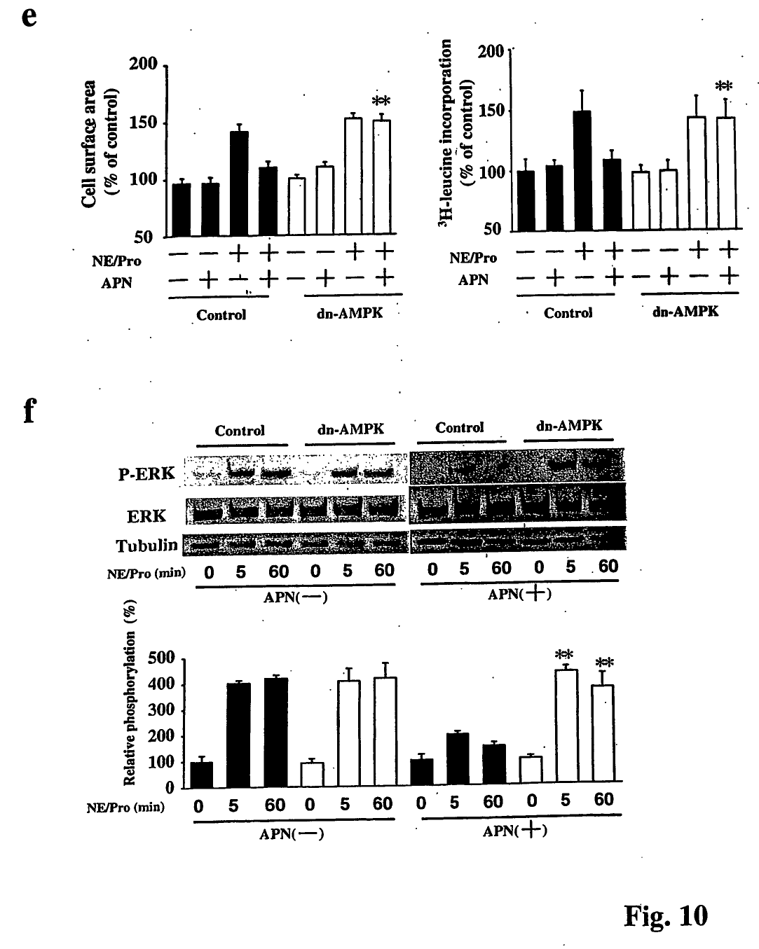

Because adiponectin functions to induce AMP-activated protein kinase (AMPK) signaling in multiple cell types including skeletal muscle, liver, adipocytes and endothelial cells^{31,64,46}, the phosphorylation of AMPK at Thr 172 of the α subunit was assessed by Western blotting. Treatment with a physiological concentration of adiponectin stimulated the phosphorylation of AMPK in cultured cardiac myocytes in a time-dependent manner (FIG. 10a). Among the three oligomeric forms of adiponectin, only the trimer stimulated AMPK phosphorylation (FIG. 10b). Conversely, AMPK phosphorylation was attenuated in APN-KO compared to WT hearts in both sham operation and TAC conditions (FIG. 10c). To test whether AMPK is involved in the inhibitory effects of adiponectin on myocyte hypertrophy, cultured cardiac myocytes were transduced with an adenoviral vector expressing a c-Myc-tagged dominant-negative mutant of AMPK (Ad-dnAMPK). Transduction with Ad-dnAMPK suppressed adiponectin-induced AMPK phosphorylation and Acetyl CoA Carboxylase (ACC) phosphorylation (FIG. 10d). Quantitative measurements of multiple blots revealed that Ad-dnAMPK reduced AMPK and ACC phosphorylation by 96.7±4.2% and 89.6±4.3%, respectively, at the 60 min time point (p<0.01 vs. control). Transduction with Ad-dnAMPK also prevented the inhibitory effect of exogenous adiponectin on αAR-stimulated myocyte hypertrophy and ERK phosphorylation (FIG. 10e and FIG. 10f, respectively). Ad-dnAMPK alone had no effect on myocyte size, protein synthesis or ERK phosphorylation. Collectively, these data indicate that adiponectin exerts its inhibitory effect on hypertrophic signaling via activation of AMPK.

The present study demonstrates that the fat-derived humoral factor adiponectin can modulate cardiac remodeling. Concentric hypertrophy and diastolic dysfunction are frequently observed in diabetes and other obesity-related disorders that are associated with hypoadiponectinemia^{53,55-57}. The findings reported here indicate that hypoadiponectinemia contributes to the development of pathologic cardiac hypertrophy in such patients, and that methods to restore or increase plasma adiponectin levels are beneficial for the prevention of pathological cardiac remodeling in disorders associated with obesity. These findings can also explain why both elevated leptin levels in patients and leptin-deficiency in ob/ob mice are associated with cardiac hypertrophy^{67,68}. In each case, perturbation in leptin signaling will promote obesity and reduce adiponectin expression^{53,69}, and may thereby contribute to cardiac hypertrophy.

The ability of adiponectin to attenuate cardiac hypertrophy is likely due to its ability to stimulate AMPK-dependent signaling within cardiac myocytes⁷⁰. AMPK is a stress-activated protein kinase that participates in the regulation of energy and metabolic homeostasis^{27,28,71}. AMPK activity is increased during acute and chronic stresses such as hypoxia, ischemia and cardiac hypertrophy^{27,28,71-72}. Adiponectin can also stimulate AMPK signaling in endothelial cells^{63,73}, but no difference in capillary density was seen between WT and APN-KO hearts after TAC (data not shown) suggesting that changes in myocyte signaling mediate the cardioprotective actions of adiponectin. In cardiac myocytes, adiponectin-stimulated AMPK activation suppressed ERK activation, an important pro-hypertrophic signaling step^{61,62,74}. It has also been shown that AMPK stimulation suppresses insulin-like growth factor 1-dependent ERK phosphorylation in 3T3 cells⁷⁵. Therefore, AMPK-mediated suppression of ERK signaling has a role in the beneficial actions of adiponectin on cardiac hypertrophy and may occur in multiple tissues.

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

### REFERENCES

The following references and all others cited in the specification are incorporated by reference.
1. Zhang, Y., Proenca, R., Maffei, M., Barone, M., Leopold, L., and Friedman, J. M. (1994) Nature 372, 425-432
2. Hotamisligil, G. S., Shargill, N. S., and Spiegelman, B. M. (1993) Science 259, 87-91
3. Shimomura, I., Funahashi, T., Takahashi, M., Maeda, K., Kotani, K., Nakamura, T., Yamashita, S., Miura, M., Fukuda, Y., Takemura, K., Tokunaga, K., and Matsuzawa, Y. (1996) Nat. Med. 2, 800-803
4. Trayhurn, P., and Beattie, J. H. (2001) Proc. Nutr. Soc. 60, 329-339
5. Maeda, K., Okubo, K., Shimomura, I., Funahashi, T., Matsuzawa, Y., and Matsubara, K. (1996) Biochem. Biophys. Res. Commun. 221, 286-289
6. Arita, Y., Kihara, S., Ouchi, N., Takahashi, M., Maeda, K., Miyagawa, J., Hotta, K., Shimomura, I., Nakamura, T., Miyaoka, K., Kuriyama, H., Nishida, M., Yamashita, S., Okubo, K., Matsubara, K., Muraguchi, M., Ohmoto, Y., Funahashi, T., and Matsuzawa, Y. (1999) Biochem Biophys Res Commun 257, 79-83
7. Ouchi, N., Kihara, S., Arita, Y., Maeda, K., Kuriyama, H., Okamoto, Y., Hotta, K., Nishida, M., Takahashi, M., Nakamura, T., Yamashita, S., Funahashi, T., and Matsuzawa, Y. (1999) Circulation 100, 2473-2476
8. Kumada, M., Kihara, S., Sumitsuji, S., Kawamoto, T., Matsumoto, S., Ouchi, N., Arita, Y., Okamoto, Y., Shimomura, I., Hiraoka, H., Nakamura, T., Funahashi, T., and Matsuzawa, Y. (2003) Arterioscler Thromb Vasc Biol 23, 85-89
9. Hotta, K., Funahashi, T., Arita, Y., Takahashi, M., Matsuda, M., Okamoto, Y., Iwahashi, H., Kuriyama, H., Ouchi, N., Maeda, K., Nishida, M., Kihara, S., Sakai, N., Nalcajima, T., Hasegawa, K., Muraguchi, M., Ohmoto, Y., Nakamura, T., Yamashita, S., Hanafusa, T., and Matsuzawa, Y. (2000) Arterioscler Thromb Vasc Biol 20, 1595-1599
10. Ouchi, N., Kihara, S., Arita, Y., Nishida, M., Matsuyama, A., Okamoto, Y., Ishigami, M., Kuriyama, H., Kishida, K., Nishizawa, H., Hotta, K., Muraguchi, M., Ohmoto, Y., Yamashita, S., Funahashi, T., and Matsuzawa, Y. (2001) Circulation 103, 1057-1063
11. Arita, Y., Kihara, S., Ouchi, N., Maeda, K., Kuriyama, H., Okamoto, Y., Kumada, M., Hotta, K., Nishida, M., Takahashi, M., Nakamura, T., Shimomura, I., Muraguchi, M., Ohmoto, Y., Funahashi, T., and Matsuzawa, Y. (2002) Circulation 105, 2893-2898
12. Okamoto, Y., Kihara, S., Ouchi, N., Nishida, M., Arita, Y., Kumada, M., Ohashi, K., Sakai, N., Shimomura, I., Kobayashi, H., Terasaka, N., Inaba, T., Funahashi, T., and Matsuzawa, Y. (2002) Circulation 106, 2767-2770
13. Matsuda, M., Shimomura, I., Sata, M., Arita, Y., Nishida, M., Maeda, N., Kumada, M., Okamoto, Y., Nagaretani, H., Nishizawa, H., Kishida, K., Komuro, R., Ouchi, N., Kihara, S., Nagai, R., Funahashi, T., and Matsuzawa, Y. (2002) J Biol Chem 277, 37487-37491
14. Maeda, N., Shimomura, I., Kishida, K., Nishizawa, H., Matsuda, M., Nagaretani, H., Furuyama, N., Kondo, H., Takahashi, M., Arita, Y., Komuro, R., Ouchi, N., Kihara, S., Tochino, Y., Okutomi, K., Horie, M., Takeda, S., Aoyama, T., Funahashi, T., and Matsuzawa, Y. (2002) Nat. Med. 8, 731-737
15. Ferrara, N., and Alitalo, K. (1999) Nat. Med. 5, 1359-1364
16. Vita, J. A., and Loscalzo, J. (2002) Circulation 106, 164-166
17. Carmeliet, P. (2003) Nat Med 9, 653-660
18. Shiojima, I., and Walsh, K. (2002) Circ. Res. 90, 1243-1250
19. Fujio, Y., and Walsh, K. (1999) J. Biol. Chem. 274, 16349-16354
20. Kim, I., Kim, H. G., So, J.-N., Kim, J. H., Kwak, H. J., and Koh, G. Y. (2000) Circ. Res. 86, 24-29
21. Kureishi, Y., Luo, Z., Shiojima, I., Bialik, A., Fulton, D., Lefer, D. J., Sessa, W. C., and Walsh, K. (2000) Nat. Med. 6, 1004-1010
22. Fulton, D., Gratton, J. P., McCabe, T. J., Fontana, J., Fujio, Y., Walsh, K., Franke, T. F., Papapetropoulos, A., and Sessa, W. C. (1999) Nature 399, 597
23. Dimmeler, S., Fleming, I., Fisslthaler, B., Hermann, C., Busse, R., and Zeiher, A. Z. (1999) Nature 399, 601-605
24. Luo, Z., Fujio, Y., Kureishi, Y., Rudic, R. D., Daumerie, G., Fulton, D., Sessa, W. C., and Walsh, K. (2000) J. Clin. Invest. 106, 493-499
25. Morales-Ruiz, M., Fulton, G., Sowa, G., Languino, L. R., Fujio, Y., Walsh, K., and Sessa, W. C. (2000) Circ. Res. 86, 892-896
26. Mu, J., Brozinick, J. T., Jr., Valladares, O., Bucan, M., and Birnbaum, M. J. (2001) Mol. Cell. 7, 1085-1094
27. Kudo, N., Barr, A. J., Barr, R. L., Desai, S., and Lopaschuk, G. D. (1995) J. Biol. Chem. 270, 17513-17520
28. Nagata, D., Mogi, M., and Walsh, K. (2003) J Biol Chem 278, 31000-31006
29. Tomas, E., Tsao, T. S., Saha, A. K., Murrey, H. E., Zhang Cc, C., Itani, S. I., Lodish, H. F., and Ruderman, N. B. (2002) Proc Natl Acad Sci USA 99, 16309-16313
30. Yamauchi, T., Kamon, J., Minokoshi, Y., Ito, Y., Waki, H., Uchida, S., Yamashita, S., Noda, M., Kita, S., Ueki, K., Eto, K., Akanuma, Y., Froguel, P., Foufelle, F., Ferre, P., Carling, D., Kimura, S., Nagai, R., Kahn, B. B., and Kadowaki, T. (2002) Nat Med 8, 1288-1295
31. Wu, X., Motoshima, H., Mahadev, K., Stalker, T. J., Scalia, R., and Goldstein, B. J. (2003) Diabetes 52, 1355-1363
32. Chen, H., Montagnani, M., Funahashi, T., Shimomura, I., and Quon, M. J. (2003) J Biol Chem, [Epub ahead of print]
33. Ziche, M., Morbidelli, L., Masini, E., Amerini, S., Granger, H. J., Maggi, C. A., Geppetti, P., and Ledda, F. (1994) J. Clin. Invest. 94, 2036-2044
34. Phillips, G. D., Stone, A. M., Jones, B. D., Schultz, J. C., Whitehead, R. A., and Knighton, D. R. (1994) In Vivo 8, 961-965
35. Chen, Z. P., Mitchelhill, K. I., Michell, B. J., Stapleton, D., Rodriguez-Crespo, I., Witters, L. A., Power, D. A., Ortiz de Montellano, P. R., and Kemp, B. E. (1999) FEBS Lett. 443, 285-289
36. Morrow, V. A., Foufelle, F., Connell, J. M., Petrie, J. R., Gould, G. W., and Salt, I. P. (2003) J Biol Chem 278, 31629-31639
37. Kovacic, S., Soltys, C. L., Barr, A. J., Shiojima, I., Walsh, K., and Dyck, J. R. (2003) J Biol Chem
38. Yamauchi, T., Kamon, J., Ito, Y., Tsuchida, A., Yokomizo, T., Kita, S., Sugiyama, T., Miyagishi, M., Hara, K., Tsunoda, M., Murakami, K., Ohteki, T., Uchida, S., Takekawa, S., Waki, H., Tsuno, N. H., Shibata, Y., Terauchi, Y., Froguel, P., To be, K., Koyasu, S., Taira, K., Kitamura, T., Shimizu, T., Nagai, R., and Kadowaki, T. (2003) Nature 423, 762-769
39. Jakobsen, S. N., Hardie, D. G., Morrice, N., and Tomqvist, H. E. (2001) J. Biol. Chem. 276, 46912-46916
40. Yamauchi, T., Kamon, J., Waki, H., Terauchi, Y., Kubota, N., Hara, K., Mori, Y., Ide, T., Murakami, K., Tsuboyama-Kasaoka, N., Ezaki, O., Akanuma, Y., Gavrilova, O., Vinson, C., Reitman, M. L., Kagechika, H., Shudo, K., Yoda, M., Nakano, Y., To be, K., Nagai, R., Kimura, S., Tomita, M., Froguel, P., and Kadowaki, T. (2001) Nat Med 7, 941-946
41. Milan, G., Granzotto, M., Scarda, A., Calcagno, A., Pagano, C., Federspil, G., and Vettor, R. (2002) Obes Res 10, 1095-1103
42. Waltenberger, J. (2001) Cardiovasc Res 49, 554-560
43. Schaper, W., and Buschmann, I. (1999) Circulation 99, 2224-2226
44. Rivard, A., Silver, M., Chen, D., Kearney, M., Magner, M., Annex, B., Peters, K., and Isner, J. M. (1999) Am J Pathol 154, 355-363
45. Janiak, P., Lainee, P., Grataloup, Y., Luyt, C. E., Bidouard, J. P., Michel, J. B., O'Connor, S. E., and Herbert, J. M. (2002) Cardiovasc Res 56, 293-302
46. Folkman and Shing, (1992) J. Biol. Chem. 267 (16), 10931-10934
47. Folkman, (1971) N. Engl. J. Med., 285:1182-1186
48. Yanagisawa-Miwa, et al., (1992) Science, 257:1401-1403
49. Baffour, et al., (1992) J Vasc Surg, 16:181-91
50. Takeshita, et al., (1994) Circulation, 90:228-234
51. Takeshita, et al., (1994) J Clin Invest, 93:662-70
52. Reilly, M. P. & Rader, D. J. Circulation 108, 1546-1551 (2003).
53. Ouchi, N., Kihara, S., Funahashi, T., Matsuzawa, Y. & Walsh, K. Obesity, adiponectin and vascular inflammatory disease. Curr Opin Lipidol 14, 561-566 (2003).
54. Kahn, B. B. & Flier, J. S. J Clin Invest 106, 473-481 (2000).
55. Ilercil, A. et al. Am Heart J 141, 992-998 (2001).
56. Rutter, M. K. et al. Circulation 107, 448-454 (2003).
57. Schannwell, C. M., Schneppenheim, M., Perings, S., Plehn, G. & Strauer, B. E. Cardiology 98, 33-39 (2002).
58. Scherer, P. E., Williams, S., Fogliano, M., Baldini, G. & Lodish, H. F. J Biol Chem 270, 26746-26749 (1995).
59. Maeda, N. et al. Nat. Med. 8, 731-737 (2002).
60. Kubota, N. et al. J Biol Chem 277, 25863-25866 (2002).
61. Xiao, L. et al. J Mol Cell Cardiol 33, 779-787 (2001).
62. Esposito, G. et al. Circulation 103, 1453-1458 (2001).
63. Kobayashi, H. et al. Circ Res 94, e27-31 (2004).
64. Tomas, E. et al. Proc Natl Acad Sci USA 99, 16309-16313 (2002).
65. Yamauchi, T. et al. Nat Med 8, 1288-1295 (2002).
66. Ouchi, N. et al. J Biol Chem 279, 1304-1309 (2004).
67. Paolisso, G. et al. Hypertension 34, 1047-1052 (1999).
68. Barouch, L. A., Berkowitz, D. E., Harrison, R. W., O'Donnell, C. P. & Hare, J. M. Circulation 108, 754-759 (2003).
69. Hu, E., Liang, P. & Spiegelman, B. M. J Biol Chem 271, 10697-10703 (1996).
70. Chan, A. Y., Soltys, C. L., Young, M. E., Proud, C. G. & Dyck, J. R. J Biol Chem, epub ahead of print (2004).
71. Mu, J., Brozinick, J. T., Jr., Valladares, O., Bucan, M. & Birnbaum, M. J. Mol. Cell. 7, 1085-1094 (2001).
72. Tian, R., Musi, N., D'Agostino, J., Hirshman, M. F. & Goodyear, L. J. Circulation 104, 1664-1669 (2001).
73. Shibata, R. et al. J Biol Chem, epub ahead of print (2004).
74. Pimentel, D. R et al. Circ Res 89, 453-460 (2001).
75. Kim, J. et al. J Biol Chem 276, 19102-19110 (2001).
76. Rogers, J. H. et al. J Clin Invest 104, 567-576 (1999).
   All references described herein and throughout the specification are incorporated herein by reference in their entirety.

We claim:

1. A method for stimulating angiogenesis in a tissue comprising administering to said tissue an angiogenesis stimulating amount of a pharmaceutical composition comprising an adiponectin protein or angiogenic stimulating portion thereof in a pharmaceutically acceptable carrier or a nucleic acid cassette containing a nucleic acid encoding for adiponectin or angiogeneic stimulating portion thereof operably linked to promoter.

2. The method of claim 1, wherein the promoter is inducible.

3. The method of claim 1, wherein said tissue has a disease or disorder associated with insufficient angiogenesis.

4. The method of claim 3, wherein the disease or disorder results in abnormal circulation.

5. The method of claim 3, wherein said tissue is myocardial.

6. The method of claim 3, wherein said disorder is a cardiac disorder.

7. The method of claim 1, wherein the adiponectin protein is a trimer.

8. A method for treating a cardiac disorder comprising administering to a patient having said disorder a pharmaceutical composition comprising adiponectin protein or effective portion thereof or a nucleic acid cassette containing a nucleotide sequence encoding adiponectin or effective portion thereof, operably linked to a promoter.

9. The method of claim 8, wherein said cardiac disorder is associated with abnormal circulation.

10. The method of claim 8, wherein said cardiac disorder is cardiac myopathy.

11. The method of claim 8, wherein the adiponectin protein is a trimer.

12. The method of claim 8, wherein the promoter is inducible.

13. A kit for stimulating angiogenesis or treating a cardiac disorder in a target mammalian tissue comprising a composition comprising a nucleic acid cassette containing a nucleic acid segment encoding for adiponectin operably linked to a promoter or a pharmaceutical composition of adiponectin protein or effective portion thereof and a pharmaceutically acceptable carrier or excipient.

14. The kit of claim 13, wherein said tissue has a disease or disorder associated with insufficient angiogenesis.

15. The kit of claim 14, wherein the disorder is a cardiac disorder.

16. The kit of claim 13, wherein the cardiac disorder is selected from the group consisting of myocardial infarction, cardiac hypertrophy, and cardiac myopathy.

17. (canceled)

18. (canceled)

19. (canceled)

20. (canceled)

21. The method of claim 3, wherein the disease or disorder is selected from the group consisting of a hypoxic tissue, an ulcer, an ischemic limb, diabetes, ischemic bowel syndrome, impotence, peripheral vascular disease, vasculitis, ischemic vascular disease, peripheral arterial occlusive disease, cerebrovascular disease and wound.

22. The method of claim 10, wherein the cardiac myopathy is ischemic.

23. The method of claim 22, wherein the ischemic cardiac myopathy is selected from the group consisting of myocardial infarct, atherosclerosis, coronary artery disease, and coronary artery insufficiency.

24. The method of claim 10, wherein the cardiac myopathy is cardiac hypertrophy.
